(19) United States
(12) Patent Application Publication
(10) Pub. No.: US 2026/0021244 A1
(43) Pub. Date: Jan. 22, 2026

(54) MEDICAL FLUID TRANSFER SYSTEMS AND ASSOCIATED METHODS

(71) Applicant: Jay Hachey, Laguna Hills, CA (US)

(72) Inventor: Jay Hachey, Laguna Hills, CA (US)

(21) Appl. No.: 19/340,488

(22) Filed: Sep. 25, 2025

Related U.S. Application Data
(63) Continuation of application No. 19/228,639, filed on Jun. 4, 2025.
(60) Provisional application No. 63/656,445, filed on Jun. 5, 2024.
(60) Provisional application No. 63/656,453, filed on Jun. 5, 2024.
(60) Provisional application No. 63/656,455, filed on Jun. 5, 2024.

(51) Int. Cl. A61M 5/14; A61J 1/20; A61M 5/142; A61M 5/168

(52) U.S. Cl. CPC A61M 5/1409; A61M 5/14216; A61M 5/16827; A61J 1/2096

(57) ABSTRACT

A medical fluid transfer system can include a base, a first channel pump, a second channel pump, and a controller. The first channel pump is configured to convey a first medical fluid from a first fluid source to a fluid receiver. The second channel pump is configured to convey a second medical fluid from a second fluid source to the fluid receiver. Each of the first channel pump and the second channel pump includes a respective cassette with a pump reservoir, a plunger body, and a pump driver. A method of operating a medical fluid transfer system can include transferring a first medical fluid and a second medical fluid to a fluid receiver at least partially based on a user input representing a first predetermined volume of the first medical fluid and a second predetermined volume of the second medical fluid to be transferred to a fluid receiver.

### CROSS REFERENCE TO RELATED APPLICATIONS

The present application is a continuation of U.S. patent application Ser. No. 19/228,639, filed on Jun. 4, 2025, and claims the benefit of U.S. Provisional Patent Application Nos. 63/656,445, 63/656,453, and 63/656,455, each filed on Jun. 5, 2024. The entire disclosures of the related applications are incorporated by reference herein.

### FIELD

The present disclosure relates generally to medical fluid transfer systems, specifically to systems for preparing medical fluids to be administered to a patient.

### BACKGROUND

When a patient is in need of medication or fluid therapy, a clinician can prepare a fluid formulation that includes one or more fluids to be administered to the patient. The fluid formulation can be specifically prepared for the needs of the patient, such as to have a specified total volume and/or to include a plurality of fluids mixed in a specified ratio. As a more specific example, the fluid formulation can include a specified dosage of a drug in combination with a bulk carrier fluid (e.g., a saline solution).

In an example, a clinician can prepare a fluid formulation by delivering medication to a pre-filled bag. In such an example, the clinician will obtain a pre-filled medication bag as well as a drug to push into medication bag, such as with a syringe. The clinician will practice aseptic techniques that may include wiping a bag port and/or drug septum to sterilize such surfaces. The clinician may attach accessories to the syringe, drug vial, and/or patient bag. The clinician can draw medication from the drug vial and attach the syringe to the bag port of the medication bag to deliver the medication into the medication bag. The clinician may check to ensure the volume of medication matches the desired volume. In some cases where the desired volume exceeds the volume of the syringe, a clinician may repeat the process with a new syringe assembly.

In another example, a clinician can prepare a fluid formulation by delivering a medication from a stock bag to an empty bag or syringe. In such an example, the clinician will obtain an empty medication bag or syringe (“patient container”). The clinician will obtain a container of pre-mixed drug to push into the patient container. The clinician will practice aseptic techniques that may include wiping a bag port and/or drug septum to sterilize such surfaces. The clinician may attach accessories to the syringe, drug vial, and/or patient bag. The clinician can draw medication from a pre-mixed medication container, such as with a syringe. The clinician may check to ensure the volume of medication matches the desired volume. The clinician can attach the syringe to the bag port of the patient container and dispense the volume of medication in the syringe into the patient container. In some cases where the desired volume exceeds the volume of the syringe, the clinician may repeat the process with a new syringe assembly.

The manual procedures outlined above, however, can be susceptible to human error. Each year in the United States alone, 7,000 to 9,000 people die as a result of medication error. Additionally, hundreds of thousands of other patients experience but often do not report an adverse reaction or other medication complications. The total cost associated with such errors exceeds $40 billion each year. Accordingly, single-channel electronic compounding devices may be used to automate various aspects of such procedures. Traditional compounding devices, however, can be limited in their accuracy, efficiency, and/or versatility.

### SUMMARY

Medical fluid transfer systems and associated methods are disclosed herein. In a representative example, a medical fluid transfer system includes a base, a first channel pump supported by the base and comprising a first inlet port and a first outlet port, a second channel pump supported by the base and comprising a second inlet port and a second outlet port, and a controller programmed to at least partially control operation of the medical fluid transfer system. The first inlet port is configured to be fluidly coupled to a first fluid source containing a first medical fluid. The second inlet port is configured to be fluidly coupled to a second fluid source containing a second medical fluid. The first channel pump is configured to convey a subset of the first medical fluid from the first fluid source to a fluid receiver that is fluidly coupled to each of the first outlet port and the second outlet port. The second channel pump is configured to convey a subset of the second medical fluid from the second fluid source to the fluid receiver. The controller is programmed to control operation of the medical fluid transfer system such that, in a first mode of operation, the controller at least partially controls operation of each of the first channel pump and the second channel pump such that the first channel pump conveys a first predetermined volume of the first medical fluid from the first fluid source to the fluid receiver and such that the second channel pump conveys a second predetermined volume of the second medical fluid from the second fluid source to the fluid receiver. The first channel pump includes a first cassette comprising a first pump reservoir, a first plunger body, and a first pump driver configured to drive the first pump driver within the first pump reservoir to vary a volume of the first pump reservoir. The second channel pump comprises a second cassette comprising a second pump reservoir, a second plunger body, and a second pump driver configured to drive the second pump driver within the second pump reservoir to vary a volume of the second pump reservoir.

In another representative example, a method of operating a medical fluid transfer system to transfer a first medical fluid from a first fluid source to a fluid receiver and to transfer a second medical fluid from a second fluid source to the fluid receiver includes receiving, with a controller of the medical fluid transfer system, a user input representing each of a first predetermined volume of the first medical fluid to be transferred from the first fluid source to the fluid receiver and a second predetermined volume of the second medical fluid to be transferred from the second fluid source to the fluid receiver. The method additionally includes withdrawing the first medical fluid from the first fluid source into a first channel pump of the medical fluid transfer system, transferring the first medical fluid from the first channel pump to the fluid receiver, withdrawing the second medical fluid from the second fluid source into a second channel pump of the medical fluid transfer system, and transferring the second medical fluid from the second channel pump to the fluid receiver. The withdrawing the first medical fluid from the first fluid source into the first channel pump comprises operating a first pump driver of the first channel pump to perform a first intake stroke to draw the first medical fluid into a first pump reservoir of the first channel pump. The transferring the first medical fluid from the first channel pump to the fluid receiver comprises operating the first pump driver to perform a first expulsion stroke to transfer the first medical fluid from the first pump reservoir to the fluid receiver. The withdrawing the second medical fluid from the second fluid source into the second channel pump comprises operating a second pump driver of the second channel pump to perform a second intake stroke to draw the second medical fluid into a second pump reservoir of the second channel pump. The transferring the second medical fluid from the second channel pump to the fluid receiver comprises operating the second pump driver to perform a second expulsion stroke to transfer the second medical fluid from the second pump reservoir to the fluid receiver.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a schematic illustration of a medical fluid transfer system according to an example.

FIG. 2A is an illustration of a medical fluid transfer system according to another example.

FIG. 2B is an illustration of a first channel pump of the medical fluid transfer system of FIG. 2A.

FIG. 3 is an illustration of a medical fluid transfer system according to another example.

FIG. 4A is an illustration of a medical fluid transfer system with a second fluid conduit that is coupled to a fluid receiver via a conduit junction according to an example.

FIG. 4B is an illustration of a medical fluid transfer system with a second fluid conduit that is directly coupled to a fluid receiver according to an example.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “operatively coupled,” as used to describe a configuration and/or relationship between two or more components, is intended to refer to a configuration and/or relationship in which the components are directly or indirectly coupled to one another in a manner consistent with the structures and/or functions disclosed herein. For example, a pair of components may be described as being operatively coupled to one another when such components are coupled to one another in a manner that is operative to produce the structural configurations and/or functional properties disclosed herein.

As used herein, “e.g.” means “for example,” and “i.e.” means “that is.”

Unless otherwise stated, as used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “substantially equal” refers to quantities that are fully equal, as well as to quantities that differ from one another by up to 25%.

The systems, apparatus, and methods described herein should not be construed as limiting in any way. Instead, the present disclosure is directed toward all novel and non-obvious features and aspects of the various disclosed examples, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

The innovations can be described in the general context of computer-executable instructions, such as those included in program modules, being executed in a computing system on a target real or virtual processor. Generally, program modules or components include routines, programs, libraries, objects, classes, components, data structures, etc. that perform particular tasks or implement particular abstract data types. The functionality of the program modules may be combined or split between program modules as desired in various examples. Computer-executable instructions for program modules may be executed within a local or distributed computing system. In general, a computing system or computing device can be local or distributed, and can include any combination of special-purpose hardware and/or general-purpose hardware with software implementing the functionality described herein, examples of which include personal computers, hand-held devices, tablets, multiprocessor systems, microprocessor-based or programmable consumer electronics, network PCs, minicomputers, mainframe computers, virtual machines, containerized applications, etc.

In various examples described herein, a module (e.g., component or engine) can be “programmed” and/or “coded” to perform certain operations or provide certain functionality, indicating that computer-executable instructions for the module can be executed to perform such operations, cause such operations to be performed, or to otherwise provide such functionality. Although functionality described with respect to a software component, module, or engine can be carried out as a discrete software unit (e.g., program, function, class method), it need not be implemented as a discrete unit. That is, the functionality can be incorporated into a larger or more general-purpose program, such as one or more lines of code in a larger or general-purpose program.

Described algorithms may be, for example, embodied as software or firmware instructions carried out by a digital computer. For instance, any of the disclosed methods can be performed by one or more a computers or other computing hardware that is part of a system and/or device according to the present disclosure. The computers can be computer systems comprising one or more processors (processing devices) and tangible, non-transitory computer-readable media (e.g., one or more optical media discs, volatile memory devices (such as DRAM or SRAM), or nonvolatile memory or storage devices (such as hard drives, NVRAM, and solid-state drives (e.g., Flash drives)). The one or more processors can execute computer-executable instructions stored on one or more of the tangible, non-transitory computer-readable media, and thereby perform any of the disclosed techniques. For instance, software for performing any of the disclosed examples can be stored on the one or more volatile, non-transitory computer-readable media as computer-executable instructions, which when executed by the one or more processors, cause the one or more processors to perform any of the disclosed techniques or subsets of techniques.

### Introduction to the Disclosed Technology

When a patient is in need of medication or fluid therapy, a clinician can prepare a fluid formulation that includes one or more fluids to be administered to the patient. The fluid formulation can be specifically prepared for the needs of the patient, such as to have a specified total volume and/or to include a plurality of fluids mixed in a specified ratio. As a more specific example, the fluid formulation can include a specified dosage of a drug in combination with a bulk carrier fluid (e.g., a saline solution).

In an example, a clinician can prepare a fluid formulation by delivering medication to a pre-filled bag. In such an example, the clinician will obtain a pre-filled medication bag as well as a drug to push into medication bag, such as with a syringe. The clinician will practice aseptic techniques that may include wiping a bag port and/or drug septum to sterilize such surfaces. The clinician may attach accessories to the syringe, drug vial, and/or patient bag. The clinician can draw medication from the drug vial and attach the syringe to the bag port of the medication bag to deliver the medication into the medication bag. The clinician may check to ensure the volume of medication matches the desired volume. In some cases where the desired volume exceeds the volume of the syringe, a clinician may repeat the process with a new syringe assembly.

In another example, a clinician can prepare a fluid formulation by delivering a medication from a stock bag to an empty bag or syringe. In such an example, the clinician will obtain an empty medication bag or syringe (“patient container”). The clinician will obtain a container of pre-mixed drug to push into the patient container. The clinician will practice aseptic techniques that may include wiping a bag port and/or drug septum to sterilize such surfaces. The clinician may attach accessories to the syringe, drug vial, and/or patient bag. The clinician can draw medication from a pre-mixed medication container, such as with a syringe. The clinician may check to ensure the volume of medication matches the desired volume. The clinician can attach the syringe to the bag port of the patient container and dispense the volume of medication in the syringe into the patient container. In some cases where the desired volume exceeds the volume of the syringe, the clinician may repeat the process with a new syringe assembly.

The manual procedures outlined above, however, can be susceptible to human error. Each year in the United States alone, 7,000 to 9,000 people die as a result of medication error. Additionally, hundreds of thousands of other patients experience but often do not report an adverse reaction or other medication complications. The total cost associated with such errors exceeds $40 billion each year. Accordingly, single-channel electronic compounding devices may be used to automate various aspects of such procedures. Traditional compounding devices, however, can be limited in their accuracy, efficiency, and/or versatility.

The present disclosure is directed to systems and methods for conveying and/or combining various medical fluids into a container from which such fluids may be administered to a patient. As discussed in more detail below, the systems and methods disclosed herein can enable preparing such fluid formulations with a high degree of speed and accuracy and with a compact apparatus.

In some examples, medical fluid transfer systems according to the present disclosure include two pumps (or channels) that can each draw a fluid from separate respective fluid sources or from the same fluid source. A controller (e.g., a computer) can operate the pumps in a coordinated manner to efficiently and accurately transfer the fluid, such as to a fluid receiver. The systems also can include a user interface (e.g., a touchscreen tablet device) via which medical staff may input the desired mixture volume and other information pertaining to a fluid transfer operation.

Medical fluid transfer systems according to the present disclosure can operate in any of a variety of use modes. For example, a system can operate as a two-channel system in which two pumps alternate to quickly and accurately transfer a single fluid to a patient container. As another example, a system can operate as a two-channel system in which each pump handles a different respective fluid (e.g., a drug and a diluent) to precisely convey a mixture of the fluids to a patient container. As another example, a system can operate in a single-channel mode when only one fluid needs to be transferred.

As discussed in more detail below, medical fluid transfer systems according to the present disclosure can include cassette-style pumps that can be easily removed and swapped and automated valves to control flow direction. The systems can be configured to generate records (e.g., logs) to track lot numbers, expiration dates, and fluid amounts for safety and compliance. The systems can allow a user to specify flow rates for each fluid transfer procedure. The systems can be configured to operate in conjunction with a variety of forms of fluid containers such as syringes, IV bags, drug vials, and/or bottles. Various aspects of the medical fluid systems disclosed herein can offer modular and/or disposable features, such as to allow components to be replaced, re-used, and/or optimally configured using ISO standard Luer-based connections for both hazardous and non-hazardous drugs.

### EXAMPLES OF THE DISCLOSED TECHNOLOGY

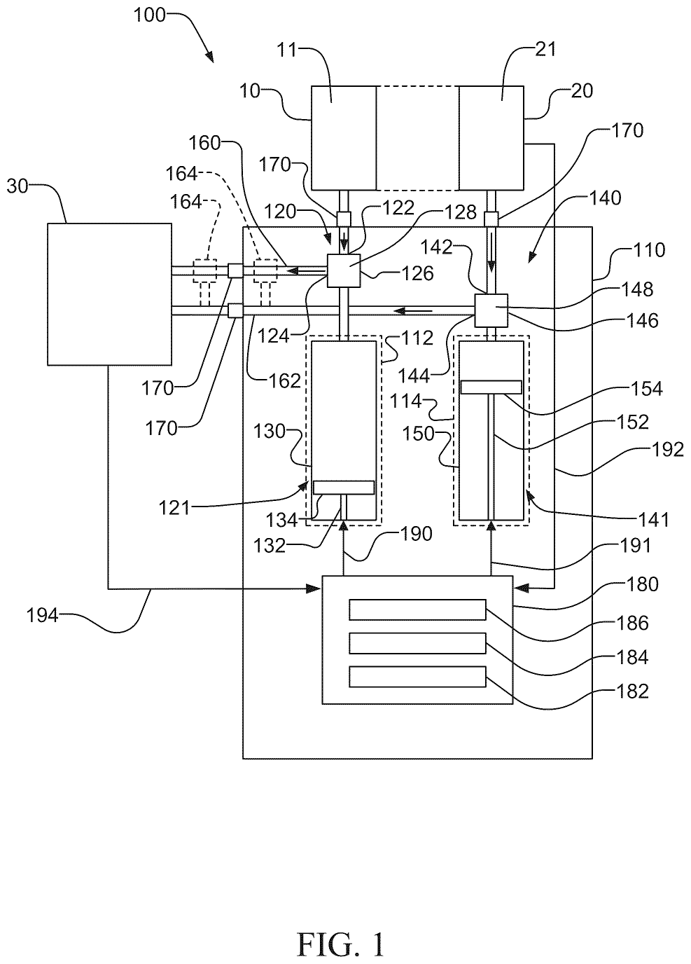

FIG. 1 schematically illustrates an example of a medical fluid transfer system 100 that can operate to convey fluids from one or more fluid sources to a fluid receiver 30, such as to prepare a fluid formulation for administration to a patient. As shown in FIG. 1, the medical fluid transfer system 100 can be configured to convey a first fluid 11 stored in a first fluid source 10 and a second fluid 21 stored in a second fluid source 20 to a fluid receiver 30. Specifically, the medical fluid transfer system 100 includes a first channel pump 120 fluidly connected to the first fluid source 10 to transfer the first fluid 11 to the fluid receiver 30 as well as a second channel pump 140 fluidly connected to the second fluid source 20 to transfer the second fluid 21 to the fluid receiver 30. The medical fluid transfer system 100 can include a base 110 that supports and/or encloses the first channel pump 120 and the second channel pump 140.

As described in more detail below, the medical fluid transfer system 100 can operate to prepare a fluid formulation in the fluid receiver 30 that includes fluids from the first fluid source 10 and the second fluid source 20 in precisely controlled proportions and/or with precisely controlled respective volumes. For example, the first fluid source 10 and/or the second fluid source 20 can contain a drug to be administered to the patient. Additionally, or alternatively, the first fluid source 10 and/or the second fluid source 20 can contain a bulk fluid (e.g., a saline solution) that can serve as a carrier fluid for a drug to be administered to the patient. As a more specific example, the first fluid source 10 can contain a drug to be administered to the patient (e.g., intravenously) and the second fluid source 20 can contain a carrier fluid to be mixed with the drug in the fluid receiver 30 for administering the drug to the patient. The bulk fluid and/or the carrier fluid additionally or alternatively may be referred to as a diluent. Accordingly, the first fluid 11 additionally or alternatively may be referred to as a first medical fluid 11 and/or the second fluid 21 additionally or alternatively may be referred to as a second medical fluid 21. The medical fluid transfer system 100 additionally or alternatively may be referred to as a compounder 100.

The first fluid source 10 and the second fluid source 20 each can include and/or be any suitable container, vessel, receptacle, etc. for containing the respective fluids. As examples, each of the first fluid source 10 and/or the second fluid source 20 can include and/or be a drug vial, a bulk solution container, a syringe, an IV bag (e.g., a two-port ethyl vinyl acetate container), etc.

Similarly, the fluid receiver 30 can include and/or be any suitable container, vessel, receptacle, etc. for containing the fluids delivered to the fluid receiver 30 by the medical fluid transfer system 100. As examples, the fluid receiver 30 can include and/or be a syringe, an IV bag, a patient bag, a bottle etc. In various examples, the fluid receiver 30 is a container from which fluids can be administered directly to a patient. Accordingly, the fluid receiver 30 additionally or alternatively may be referred to as a patient container 30. In various examples, the fluid receiver 30 can be pre-filled or empty prior to receiving fluids from the first fluid source 10 and/or the second fluid source 20 as described herein.

In some examples, and as described in more detail below, the medical fluid transfer system 100 can be configured to convey only the first fluid 11 from the first fluid source 10 to the fluid receiver 30 without additionally conveying a different second fluid 21 to the fluid receiver 30. For example, and as shown in dashed lines in FIG. 1, each of the first channel pump 120 and the second channel pump 140 can be configured to draw the first fluid 11 from two separate outlets of the first fluid source 10. Such examples can enable transferring the first fluid 11 into the fluid receiver 30 at a faster rate than when using only one channel pump. As another example, and as described in more detail below, the medical fluid transfer system 100 can operate to convey the first fluid 11 from the first fluid source 10 to the fluid receiver 30 with the first channel pump 120; e.g., without use of the second channel pump 140 and without connecting the second fluid source 20 to the medical fluid transfer system 100.

The medical fluid transfer system 100 generally is configured to be selectively and repeatedly coupled to each of the first fluid source 10, the second fluid source 20, and the fluid receiver 30. For example, and as shown in FIG. 1, the first fluid source 10, the second fluid source 20, and/or the fluid receiver 30 can be fluidly connected to the medical fluid transfer system 100 via corresponding connection ports 170. Each connection port 170 can allow for corresponding components to be selectively and repeatedly fluidly connected to one another. As examples, each connection port 170 can include and/or be a friction-fit connection port, a threaded connection port, a bayonet lock connection port, a Luer based connection, etc. In various examples, each connection port 170 has a form and/or structure that is consistent with the International Standards Organization (ISO) to ensure compatibility with other medical device accessories.

As shown in FIG. 1, each connection port 170 can be attached to (e.g., mounted on) the base 110, such as to provide inlets and outlets for fluid flow into and out of components contained within and/or supported by the base 110. This is not required, however, and it additionally is within the scope of the present disclosure that each connection port 170 may be located at any other suitable location, such as a location fully exterior of the base 110.

As discussed above, the first channel pump 120 and the second channel pump 140 are configured to convey the first fluid 11 and the second fluid 21 to the fluid receiver 30. As shown in FIG. 1, the first channel pump 120 includes an inlet port 122 that receives the first fluid 11 from the first fluid source 10 and an outlet port 124 for conveying the first fluid 11 to the fluid receiver 30. Similarly, the second channel pump 140 includes an inlet port 142 that receives the second fluid 21 from the second fluid source 20 and an outlet port 144 for conveying the second fluid 21 to the fluid receiver 30. In this manner, each of the first channel pump 120 and the second channel pump 140 may be described as representing an intermediate device for pumping fluid from one location (e.g., a container) to another location (e.g., another container).

Utilizing two channels when filling a single fluid receiver 30 in this manner can result in higher aggregate accuracy of two ingredient pharmacy admixtures. As such, the use of dual channel automated compounders such as the medical fluid transfer system 100 can offer a user the ability to enter desired volumes for two ingredients of standard compounds. This can allow the system to ensure the accuracy of both the drug and diluent when filling into an empty patient container rather than rely on pre-mixed bags that are often overfilled. Such overfill can affect the total volume accuracy of two ingredient compounds which can cause significant workflow issues when administering IV fluids.

In the example of FIG. 1, the first channel pump 120 includes a pump reservoir 130 and a pump driver 132 configured to draw the first fluid 11 into the pump reservoir 130 and to expel the first fluid 11 out of the pump reservoir 130. The first channel pump 120 additionally includes a valve 126 configured to selectively direct the first fluid 11 from the first fluid source 10 into the pump reservoir 130 or from the pump reservoir 130 into the fluid receiver 30. The valve 126 can include and/or be any of a variety of valve mechanisms for selectively and/or mechanically directing a flow of the first fluid 11, examples of which may include a stopcock, a ball valve, or a butterfly valve. Additionally, or alternatively, the valve 126 can include and/or be various mechanisms for restricting a flow of the first fluid 11 in an unintended direction, such as one or more check valves 128.

In the example of FIG. 1, the first channel pump 120 may be described as operating similarly to a piston and/or a syringe. In particular, in this example, the pump reservoir can be a generally cylindrical reservoir and the pump driver 132 can operate to drive a plunger body 134 within the pump reservoir 130 to selectively expand or contract a volume of the pump reservoir 130. Expanding the volume of the pump reservoir 130 with the plunger body 134 in this manner can operate to draw the first fluid 11 into the pump reservoir 130 from the first fluid source 10 via the valve 126, while contracting the volume of the pump reservoir 130 with the plunger body 134 can operate to expel the first fluid 11 from the pump reservoir 130 into the fluid receiver 30 via the valve 126. In such examples, the pump reservoir 130 additionally or alternatively may be referred to as a pump barrel 130 and/or as a syringe barrel 130. The pump reservoir 130 may be configured to contain a precisely defined volume of the first fluid 11 when the pump driver 132 operates to fill the pump reservoir 130. In this manner, a total volume of the first fluid 11 conveyed by the first channel pump 120 may be correlated to (e.g., determined based on) a number of times the pump driver 132 operates to fill and empty the pump reservoir 130. The second channel pump 140 can operate in an analogous and/or identical manner.

In the present disclosure, operation of the first channel pump 120 to draw the first fluid 11 out of the first fluid source 10 may be referred to as an intake stroke of the first channel pump 120, while operation of the first channel pump 120 to push the first fluid 11 into the fluid receiver 30 may be referred to as an expulsion stroke of the first channel pump 120.

In various examples, one or more components of the first channel pump 120 additionally or alternatively may be referred to as a first cassette 121, and one or more components of the second channel pump 140 additionally or alternatively may be referred to as a second cassette 141. As a more specific example, the first cassette 121 can include the pump reservoir 130 and the plunger body 134 of the first channel pump 120, and the second cassette 141 can include the pump reservoir 150 and the plunger body 154 of the second channel pump 140. In some examples, the first cassette 121 additionally includes the valve 126 and the second cassette 141 includes the valve 146.

In some examples, various components of the first channel pump 120 and/or the second channel pump 140 may be selectively removable from the base 110. For example, either or both of the first cassette 121 and the second cassette 141 can be configured to be selectively and repeatedly removed from and coupled to the base 110 without damage to the medical fluid transfer system 100.

Additionally, or alternatively, various components of the first channel pump 120 and/or the second channel pump 140 may be interchangeable. For example, the first cassette 121 and the second cassette 141 may be at least substantially identical in form, structure, and/or function such that the components of the second cassette 141 can alternatively be used in the first channel pump 120 and such that components of the first cassette 121 can alternatively be used in the second channel pump 140. Such a modular configuration may allow for components of the medical fluid transfer system 100 to be readily replaced, serviced, and/or repurposed as desired.

In some examples, the base 110 includes one or more features for engaging and/or receiving the first cassette 121 and/or the second cassette 141. For example, and as shown in FIG. 1, the base 110 may include a first pump receiver 112 configured to receive one or more elements of the first channel pump 120, such as the first cassette 121. In such examples, the first cassette 121 may be described as being seated into the first pump receiver 112 during operate use of the medical fluid transfer system 100. Stated differently, the first cassette 121 and/or the base 110 may be configured such that the first pump receiver 112 mechanically matches the industrial design (e.g., the shape and/or size) of the first cassette 121.

Similarly, the base 110 may include a second pump receiver 114 configured to engage and/or receive one or more elements of the second channel pump 140, such as the second cassette 141. In such examples, the second cassette 141 may be described as being seated into the second pump receiver 114 during operate use of the medical fluid transfer system 100. Stated differently, the second cassette 141 and/or the base 110 may be configured such that the second pump receiver 114 mechanically matches the industrial design of the second cassette 141.

In such examples, the first pump receiver 112 and/or the second pump receiver 114 can be configured to receive such components in any suitable manner, such as via frictional coupling, a snap-fit coupling, a locking engagement, and so forth. In various examples, each of the first pump receiver 112 and the second pump receiver 114 can include and/or be a recessed portion of the base 110 configured such that the corresponding components of the first channel pump 120 and the second channel pump 140 are at least partially received within the base 110 during operative use of the medical fluid transfer system 100. In some examples, the base 110, the first pump receiver 112, and/or the second pump receiver 114 are configured such that the first channel pump 120 and/or the second channel pump 140 are fully enclosed within the base during operative use of the medical fluid transfer system 100.

The medical fluid transfer system 100 may be configured such that one or more elements of the first channel pump 120 may be configured to be selectively and repeatedly removed from and received within the first pump receiver 112 without damage to the medical fluid transfer system 100. Similarly, the medical fluid transfer system 100 may be configured such that one or more elements of the second channel pump 140 may be configured to be selectively and repeatedly removed from and received within the second pump receiver 114 without damage to the medical fluid transfer system 100.

In some examples, the pump driver 132 of the first channel pump 120 and the pump driver 152 of the second channel pump 140 may be controlled independently of one another. For example, the pump driver 132 and the pump driver 152 can include respective motors (e.g., electric actuators and/or stepper motors) for independently controlling a flow into and out of the corresponding pump reservoir 130/150. In other examples, operation of the pump driver 132 and the pump driver 152 may be linked and/or mechanically coupled. For example, the medical fluid transfer system 100 may include a single motor that operates to drive each of the pump driver 132 and the pump driver 152, such as to stagger the intake and expulsion strokes of the corresponding pumps as described in more detail below.

In some examples, the directionality of the fluid flow within the valve 126 can be passively varied between the intake stroke and the expulsion stroke of the first channel pump 120. For example, the valve 126 can include a check valve 128 that permits a fluid flow through the inlet port 122 only in a direction toward the pump reservoir 130 and another check valve 128 that permits a fluid flow through the outlet port 124 only in a direction away from the pump reservoir 130. In such an example, the check valve 128 can operate to automatically direct the flow of the first fluid 11 as described above upon operation of the pump driver 132.

In other examples, the directionality of the fluid flow within the valve 126 can be actively controlled. For example, the valve 126 can include and/or be a ball valve mechanism with a fluid flow direction that is actively varied (e.g., with a controller) as operation of the pump driver 132 transitions between the intake stroke and the expulsion stroke.

As shown in FIG. 1, the second channel pump 140 includes an inlet port 142, an outlet port 144, a valve 146, a check valve 148, a pump reservoir 150, a pump driver 152, and a plunger body 154. Such components may share any suitable characteristics, features, attributes, etc. with similarly named components of the first channel pump 120 and thus are not described in detail here. Accordingly, any descriptions herein of the first channel pump 120 may be understood as applying to the second channel pump 140 in an analogous manner. Stated differently, in various examples, the first channel pump 120 and the second channel pump 140 may be at least substantially identical to one another in form and/or function.

In various examples, the inlet port 122 of the first channel pump 120 may be described as a first inlet port 122, the outlet port 124 of the first channel pump 120 may be described as a first outlet port 124, the valve 126 of the first channel pump 120 may be described as a first valve 126, the pump reservoir 130 of the first channel pump 120 may be described as a first pump reservoir 130, the pump driver 132 of the first channel pump 120 may be described as a first pump driver 132, and/or the plunger body 134 of the first channel pump 120 may be described as a first plunger body 134. Similarly, the inlet port 142 of the second channel pump 140 may be described as a second inlet port 142, the outlet port 144 of the second channel pump 140 may be described as a second outlet port 144, the valve 146 of the second channel pump 140 may be described as a second valve 146, the pump reservoir 150 of the second channel pump 140 may be described as a second pump reservoir 150, the pump driver 152 of the second channel pump 140 may be described as a second pump driver 152, and/or the plunger body 154 of the second channel pump 140 may be described as a second plunger body 154.

While the present disclosure generally relates to examples in which the first channel pump 120 and the second channel pump 140 operate according to a piston-like or syringe-like mechanism, this is not required of all examples. For example, it additionally is within the scope of the present disclosure that the first channel pump 120 and/or the second channel pump 140 can include and/or be any of a variety of pump mechanisms.

Additionally, while the present disclosure generally relates to examples in which the first channel pump 120 and the second channel pump 140 are at least substantially identical in form and/or function, this is not required of all examples. For example, in other examples, the first channel pump 120 and the second channel pump 140 can differ from one another in size, form, mechanism, and/or function.

As shown in FIG. 1, the medical fluid transfer system 100 may include a first fluid conduit 160 for conveying the first fluid 11 from the outlet port 124 toward and/or to the fluid receiver 30 and/or a second fluid conduit 162 for conveying the second fluid 21 from the outlet port 144 toward and/or to the fluid receiver 30. Each of the first fluid conduit 160 and the second fluid conduit 162 can include and/or be any of a variety of structures. For example, each of the first fluid conduit 160 and/or the second fluid conduit 162 can include and/or be a length of pipe and/or tubing. As another example, the first fluid conduit 160 and/or the second fluid conduit 162 may be at least partially defined by a portion of the base 110.

In some examples, and as shown in solid lines in FIG. 1, each of the first fluid conduit 160 and the second fluid conduit 162 can extend to the fluid receiver 30 (optionally via respective connection ports 170) such that the fluid receiver 30 receives the first fluid 11 and the second fluid 21 via separate respective inlets. In such examples, the first fluid 11 and the second fluid 21 may be mixed with one another only after reaching the fluid receiver 30.

In other examples, and as shown in dashed lines in FIG. 1, the first fluid conduit 160 and the second fluid conduit 162 may be fluidly coupled to one another such that the first fluid 11 and the second fluid 21 are mixed with one another prior to reaching the fluid receiver 30. In particular, in such examples, the medical fluid transfer system 100 can include a conduit junction 164 at which the first fluid conduit 160 and the second fluid conduit 162 are joined. In such examples, the conduit junction 164 can be located inside or outside the base 110.

In some examples, the first fluid conduit 160 and/or the second fluid conduit 162 may be offered in varying lengths to allow the first channel pump 120 and/or the second channel pump 140 to be fluidly connected to the fluid receiver 30 in any of a variety of configurations. While FIG. 1 illustrates each of the first fluid conduit 160 and the second fluid conduit 162 as extending at least substantially within the base 110, it also is within the scope of the present disclosure that the first fluid conduit 160 and/or the second fluid conduit 162 can extend at least partially exterior of the base 110.

Each of the first fluid conduit 160 and the second fluid conduit 162 can be configured to be fluidly connected to other components of the medical fluid transfer system 100 in any of a variety of manners. For example, the first fluid conduit 160 and/or the second fluid conduit 162 can be configured to be fluidly connected to the outlet port 124 of the first channel pump 120, the outlet port 144 of the second channel pump 140, the fluid receiver 30, the conduit junction 164, and/or a corresponding connection port 170. In some examples, such connections may be made via a removable connection interface (e.g., a friction-fit connection port, a threaded connection port, a bayonet lock connection port, a Luer based connection, etc.), such as to allow for the first fluid conduit 160 and/or the second fluid conduit 162 to be selectively removed and/or replaced.

As shown in FIG. 1, the medical fluid transfer system 100 can include a controller 180 that is programmed and/or configured to coordinate and/or control operation of various components. In the example of FIG. 1, the controller 180 includes a user interface 182 for receiving instructions from a user and/or for providing information to the user. In other examples, the user interface 182 may be separate from the controller 180 and communicatively connected to the controller 180, such as via wired and/or wireless connections. The controller 180 also includes a processor system 184 and a memory 186 storing computer-executable instructions that, when executed by the processor system 184, cause the medical fluid transfer system 100 to perform various methods and/or functions described herein. The controller 180 may be communicatively connected to any other components of the medical fluid transfer system 100 in any suitable manner, such as via wired and/or wireless connections. As shown in FIG. 1, the controller 180 may be contained within the base 110. In other examples, at least a portion of the controller 180 may be exterior of the base 110.

The user interface 182 can include any of a variety of components for interfacing with a user, such as a medical professional. For example, the user interface 182 can include a display device (e.g., a monitor) that is configured to provide the user with any of a variety of visual instructions, indications, and/or other information as described herein. The user interface 182 additionally or alternatively can include a user input device that is configured to receive a user input from the user in any of a variety of manners. For example, the user input device can include and/or be a keyboard, a mouse, a trackpad, a touch screen, a pointer device, etc. In some examples, the user interface 182 can include and/or be a touch screen that includes and/or performs as both a display device and a user input device. In the example of FIG. 1, the user interface 182 is connected to and supported by the base 110. In other examples, the user interface 182 may be separate from the base 110. For example, the user interface 182 can include and/or be a touchscreen tablet device that is separate from the base 110 and that is configured to transmit signals to and/or receive signals from the controller 180, such as via a wired and/or wireless interface.

The controller 180 can be programmed and/or configured to control operation of the medical fluid transfer system 100 in any of a variety of manners. For example, and as shown in FIG. 1, the controller 180 can be configured to generate and transmit one or more pump control signals to control operation of the first channel pump 120 and/or the second channel pump 140. For example, the controller 180 can be configured to generate and transmit a first pump control signal 190 to the pump driver 132 of the first channel pump 120 and to generate and transmit a second pump control signal 191 to the pump driver 152 of the second channel pump 140 to command the pump driver 132 and/or the pump driver 152 to operate as described above (e.g., to initiate the intake stroke or the expulsion stroke).

In some examples, and as shown in FIG. 1, the controller 180 can be programmed and/or configured to receive an input fluid signal 192 from the first fluid source 10 and/or the second fluid source 20 (and/or from a component associated with the first fluid source 10 and/or the second fluid source 20). For example, the input fluid signal 192 can include information regarding the fluid(s) contained in the first fluid source 10 and/or the second fluid source 20, such as to verify that the correct medication and/or carrier fluid is connected to the medical fluid transfer system 100. Additionally, or alternatively, the input fluid signal 192 can include information regarding a lot number and/or an expiration date of the fluid(s) contained in the first fluid source 10 and/or the second fluid source 20. Additionally, or alternatively, the input fluid signal 192 may be manually entered by a user, such as via the user interface 182.

In some examples, and as shown in FIG. 1, the controller 180 can be programmed and/or configured to receive an output fluid signal 194 from the fluid receiver 30 (and/or from a component associated with the fluid receiver 30). For example, the output fluid signal 194 can include information regarding the type of container being used as the fluid receiver 30, a maximum volumetric capacity of the fluid receiver 30, a measured mass of the fluid receiver 30 and/or the fluid contained therein, and/or a measurement of the volume of fluid transferred into the fluid receiver 30. As more specific examples, the volume of fluid transferred into the fluid receiver 30 by the medical fluid transfer system 100 may be measured via a mass of the fluid receiver 30 and/or via a flow meter positioned upstream of the fluid receiver 30. In such examples, the measured volume of fluid transferred into the fluid receiver 30 may be compared to an intended transferred volume of the fluid to provide verification of the amount of fluid transferred.

In this manner, the controller 180 can operate to collect and record fluid transfer information relating to each fluid transfer procedure, such as for pharmacy verification purposes. For example, the controller 180 can record information corresponding to the input fluid signal 192 (e.g., information regarding the first fluid 11 and/or the second fluid 21 transferred to the fluid receiver 30), information regarding the output fluid signal 194 (e.g., information regarding the amount of fluid actually transferred to the fluid receiver 30), and/or any other relevant information collected by the controller 180 and/or provided by the user. Such information can be recorded and/or stored in a log that is generated by the controller 180, which may be stored in the memory 186 and/or transferred to another controller and/or storage device.

Operation of the medical fluid transfer system 100 can include receiving a user input (e.g., via the user interface 182) in which a user can indicate a desired final volume of the first fluid 11, the second fluid 21, and/or a combined volume of the first fluid 11 and the second fluid 21 to be transferred to the fluid receiver 30. The user also can indicate whether to use one or both of the first channel pump 120 and/or the second channel pump 140 to perform the transfer. For example, when transferring a single fluid to the fluid receiver 30, the medical fluid transfer system 100 can advantageously provide for fast fluid transfer through simultaneous operation of the first channel pump 120 and the second channel pump 140 to draw from one or more containers holding such a fluid. In other examples, however, the user may prefer to select either such pump to be used alone for such transfer, such as to allow for maintenance of the remaining pump.

Operation of the medical fluid transfer system 100 additionally can including receiving medical fluid information regarding the fluids being transferred to the fluid receiver 30 in a given transfer operation. For example, the medical fluid information can include information regarding the identification of the first fluid 11 and/or the second fluid 21, and/or expiration dates and/or lot numbers corresponding to the fluid(s). Additionally, or alternatively, the medical fluid information can include photographs of the first fluid source 10, the second fluid source 20, and/or the fluid receiver 30, such as photos taken before and/or after performing the fluid transfer.

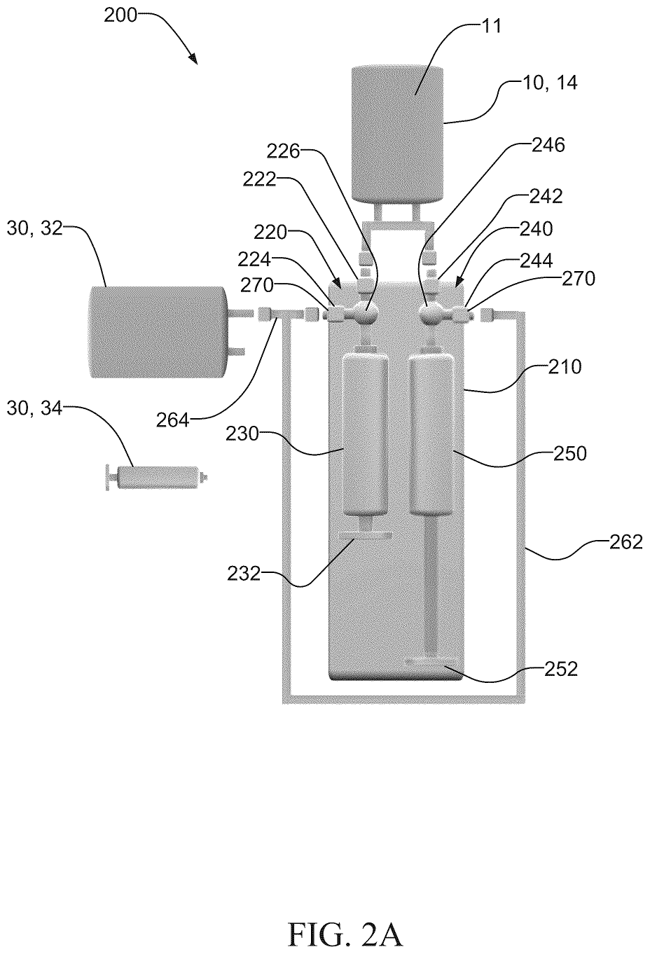

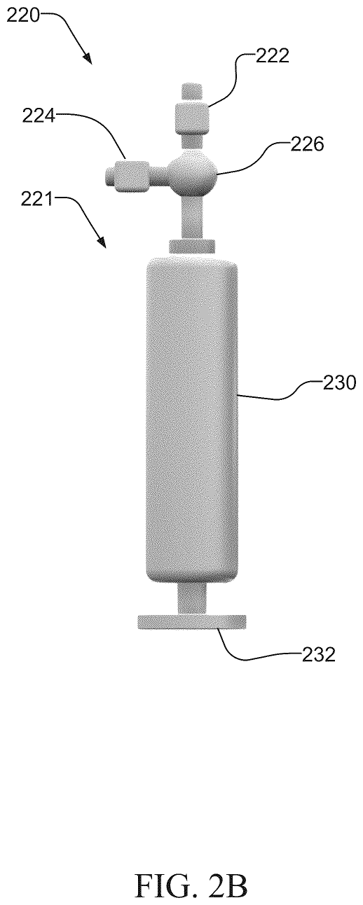

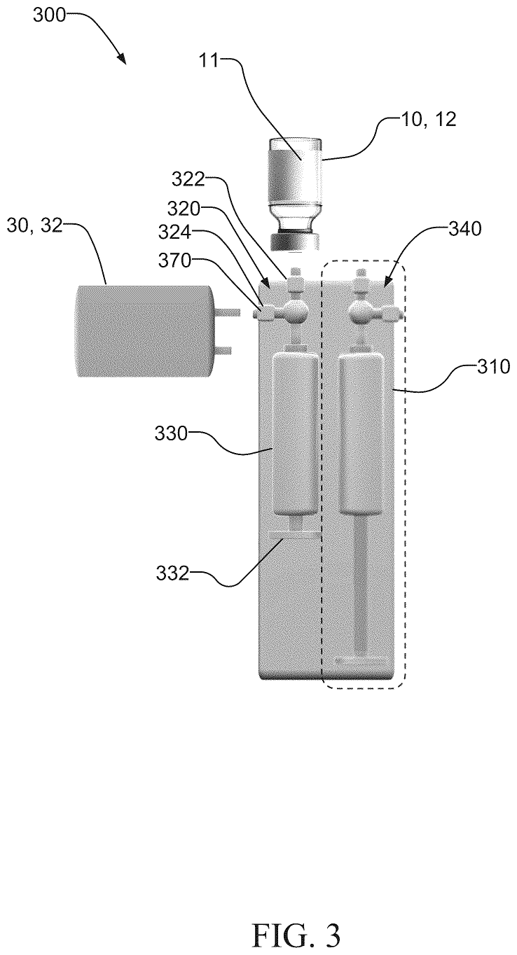

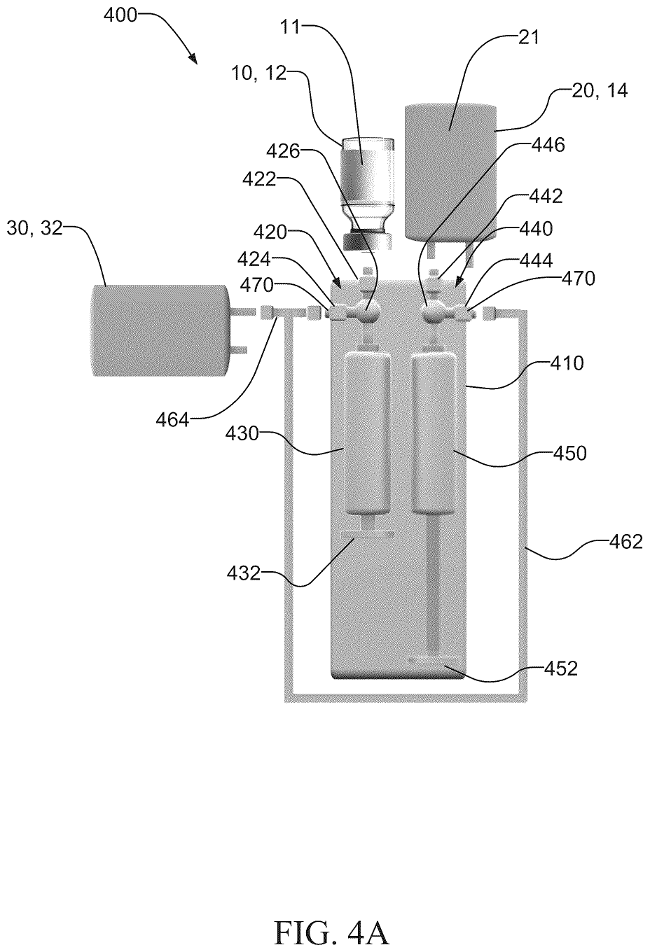

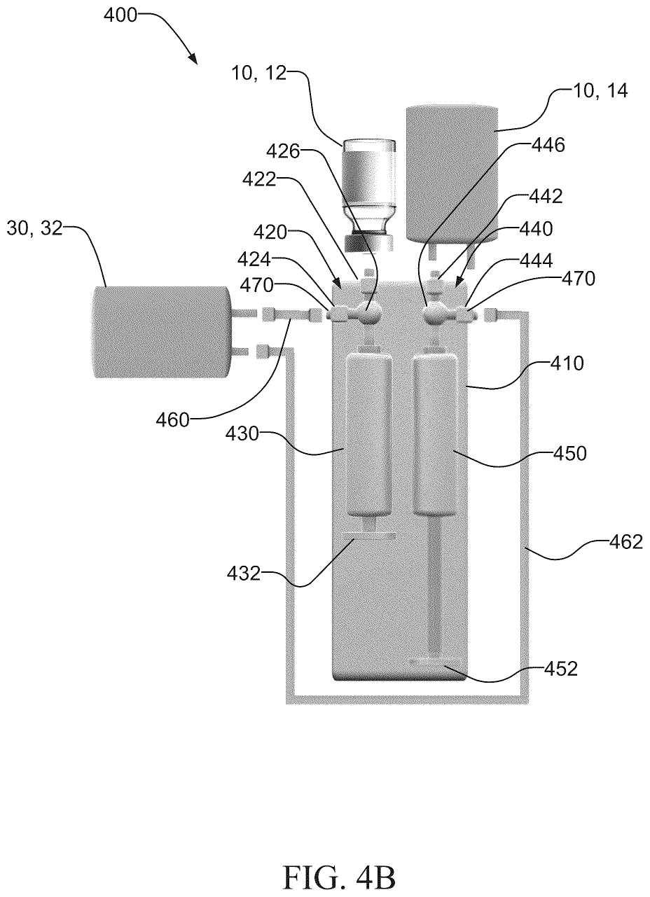

FIGS. 2A-4B represent more specific examples of medical fluid transfer systems and/or of components and operations thereof. In each of FIGS. 2A-4B, various components of the medical fluid transfer systems are labeled with reference numerals similar to those used to label corresponding components in FIG. 1. For example, the first channel pump 220 of FIGS. 2A-2B, the first channel pump 320 of FIG. 3, and the first channel pump 420 of FIGS. 4A-4B may be understood as representing examples of the first channel pump 120 of FIG. 1. Such components may not be discussed in detail with reference to each of FIGS. 2A-4B. It is to be understood that all components labeled in FIGS. 2A-4B can share any suitable characteristics, attributes, properties, functions, etc. with the corresponding components labeled in FIG. 1 and discussed above. It further is to be understood that the medical fluid transfer systems of FIGS. 2A-4B can include any other components described above with reference to FIG. 1, even when such components are not specifically illustrated or labeled in FIGS. 2A-4B.

To clarify the relationships between the constituent components of the medical fluid transfer systems of FIGS. 2A-4B, various components are illustrated as being slightly spaced apart in FIGS. 2A-4B, including components that are fluidly coupled to one another during operative use of the corresponding medical fluid transfer systems. Accordingly, it is to be understood that FIGS. 2A-4B do not necessarily depict operative configurations of the illustrated components and systems, and that the illustrated components can be fluidly connected to one another as described herein and/or as illustrated in FIG. 1 during operative use of the medical fluid transfer systems.

FIG. 2A represents an example of a medical fluid transfer system 200 in which each of the first channel pump 220 and the second channel pump 240 operates to convey the first fluid 11 from the first fluid source 10 to the fluid receiver 30. Specifically, in this example, the first fluid source 10 is a bulk fluid source 14 and the fluid receiver 30 is a medication bag 32. Operation of the medical fluid transfer system 200 thus can include transferring a subset of the contents of the bulk fluid source 14 to the medication bag 32. FIG. 2 additionally illustrates another example of a fluid receiver 30 in the form of a syringe 34 that can be connected to the medical fluid transfer system 200 in place of the medication bag 32. In other examples, the fluid receiver 30 can include and/or be a patient bag, a cassette, or a bottle.

In the example of FIG. 2A, the first fluid 11 can be an IV fluid to be administered intravenously to a patient. The first fluid 11 can include multiple ingredients, such as drug and/or diluent ingredients. In the example of FIG. 2A, the first fluid source 10 also may be referred to herein as a fluid source 10, and/or the first fluid 11 also may be referred to herein as a fluid 11.

As shown in FIG. 2A, the first fluid source 10 is fluidly connected to the respective inlet ports 222, 242 of each of the first channel pump 220 and the second channel pump 240. Such a configuration can allow for the first fluid 11 to be transferred from the first fluid source 10 to the fluid receiver 30 at a faster rate than in an example in which only one pump is used. For example, the operation of the first channel pump 220 and the second channel pump 240 can be staggered such that the first channel pump 220 draws the first fluid 11 from the first fluid source 10 through the inlet port 222 while the second channel pump 240 expels the first fluid 11 out of the outlet port 244 and into the fluid receiver 30. Stated differently, the first channel pump 220 and the second channel pump 240 can be operated such that the first channel pump 220 performs the intake stroke while the second channel pump 240 performs the expulsion stroke, and vice versa. As a result, the first fluid 11 may be continuously (or nearly continuously) drawn out of the first fluid source 10 and at the same time be continuously (or nearly continuously) driven into the fluid receiver 30.

In the example of FIG. 2A, the outlet port 224 of the first channel pump 220 is connected to the conduit junction 264 via a corresponding connection port 270 (e.g., without a corresponding first fluid conduit), while the outlet port 244 of the second channel pump 240 is connected to the conduit junction 264 via a corresponding connection port 270 and the second fluid conduit 262. The conduit junction 264 can be connected directly to the fluid receiver 30, such as via an ISO standard Luer based connection.

In an example of using the medical fluid transfer system 200 of FIG. 2A, a user (e.g., a clinician) can enter a desired amount of the first fluid 11 into a user interface of the medical fluid transfer system 200 (e.g., the user interface 182 of FIG. 1), optionally following configuration and priming of the medical fluid transfer system 200. A controller of the medical fluid transfer system 200 (e.g., the controller 180 of FIG. 1) can then operate to actuate the pump driver 232 of the first channel pump 220 to draw the first fluid 11 into the pump reservoir 230 via an intake stroke. Through a series of valves and/or stopcocks (e.g., the valve 226), the direction of the first fluid 11 can be passively and/or actively controlled to route the first fluid 11 into the fluid receiver 30, such as during a subsequent expulsion stroke of the first channel pump 220.

While the first channel pump 220 operates to transfer the first fluid 11 into the fluid receiver 30 (e.g., with an expulsion stroke), the second channel pump 240 can begin to withdraw the first fluid 11 into the pump reservoir 250 from the first fluid source 10 (e.g., with an intake stroke). In other words, while the first channel pump 220 is transferring the first fluid 11 to the fluid receiver 30, the second channel pump 240 will simultaneously withdraw the first fluid 11 from the first fluid source 10.

Similarly, while the second channel pump 240 subsequently operates to transfer the first fluid 11 into the fluid receiver 30 (e.g., with an expulsion stroke), the first channel pump 220 can begin to withdraw the first fluid 11 into the pump reservoir 230 from the first fluid source 10 (e.g., with an intake stroke). In other words, while the second channel pump 240 is transferring the first fluid 11 to the fluid receiver 30, the first channel pump 220 will simultaneously withdraw the first fluid 11 from the first fluid source 10.

In some examples, the first channel pump 220 transfers the first fluid 11 to the fluid receiver 30 at the same rate, or at a similar rate, as the second channel pump 240 withdraws the first fluid 11 from the first fluid source 10. Rates may be described as being similar to one another when the rates are within +/−10% of each other. In other examples, the rate at which the first channel pump 220 transfers the first fluid 11 to the fluid receiver 30 may be different from the rate at which the second channel pump 240 withdraws the first fluid 11 from the first fluid source 10. In some examples, the medical fluid transfer system 200 may be configured to receive (e.g., via the user interface 182 of FIG. 1) a user input specifying one or both of a first flow rate at which the first channel pump 220 transfers the first fluid 11 to the fluid receiver 30 and/or a second flow rate at which the second channel pump 240 transfers the first fluid 11 into the fluid receiver 30.

As discussed above, the medical fluid transfer system 200 can include one or more (e.g., a network of) valves and/or stopcocks, such as the valve 226 and the valve 246, to ensure that the first channel pump 220 can transfer the first fluid 11 to the fluid receiver 30 while the second channel pump 240 withdraws the first fluid 11 from the first fluid source 10 (and vice versa). For example, and as discussed above with reference to FIG. 1, the medical fluid transfer system 200 can include one or more check valves positioned and/or oriented to restrict and/or prevent a fluid flow in a direction other than those described herein. Such a configuration thus may allow for the first fluid 11 to be routed from the first fluid source 10 to the fluid receiver 30 via two separate pump channels, thus achieving expedited filling of the fluid receiver 30 relative to single-channel syringe pump designs.

Upon completion of such an operation, the fluid receiver 30 will contain an accurate volume of the first fluid 11, thus achieving accuracy for the drug volume and/or total volume in the fluid receiver 30 without relying on pre-filled IV fluid containers.

FIG. 2B illustrates the first channel pump 220 in more detail. Because the first channel pump 220 and the second channel pump 240 may be interchangeable, FIG. 2B equivalently may be described as illustrating aspects of the second channel pump 240. FIG. 2B additionally or alternatively may be described as illustrating a first cassette 221 that can be used in and/or as either of the first channel pump 220 or the second channel pump 240 of FIG. 2A. The various components of the first channel pump 220 illustrated in FIG. 2B can be coupled (e.g., bonded) together to form a singular unit with which a user (e.g., a clinician) can interact and place into the medical fluid transfer system 200.

As shown in FIG. 2B, and similar to the first channel pump 120 of FIG. 1, the first channel pump 220 includes a valve 226, a pump reservoir 230, and a pump driver 232. Various components of the first channel pump 220 may be collectively referred to as a first cassette 221. For example, the first cassette 221 can include the pump reservoir 230 and the pump driver 232, and further can include the valve 226.

The valve 226 can include and/or be any of a variety of components for actively and/or passively directing a fluid path in desired directions, such as a stopcock, one or more check valves, etc. In some examples, the valve 226 can work in conjunction with (e.g., can be at least partially controlled by) a controller, such as via a control signal generated by the controller 180 of FIG. 1.

As shown in FIG. 2B, the first channel pump 220 includes an inlet port 222 and an outlet port 224, each of which may be coupled (e.g., bonded) to the valve 226. The inlet port 222 allows a desired medical fluid to enter the first channel pump 220, while the outlet port 224 allows a desired medical fluid to exit the first channel pump 220. In various examples, the valve 226 may be described as including the inlet port 222 and/or the outlet port 224. Additionally, or alternatively, various components described herein as being optional components of the valve 226 (e.g., the check valve 128 of FIG. 1) may be a component of the inlet port 222 and/or of the outlet port 224. Each of the inlet port 222 and the outlet port 224 can include and/or be any of a variety of connections, such as an ISO-compliant male Luer based connection.

While the first channel pump 220 of FIG. 2B is described here primarily in the context of the medical fluid transfer system 200 of FIG. 2A, it is to be understood that the descriptions herein of the first channel pump 220 also can apply to any other pumps disclosed herein, such as the pumps of any of FIGS. 3-4B.

FIG. 3 represents an example of a medical fluid transfer system 300 in which the first channel pump 320 operates to convey the first fluid 11 from the first fluid source 10 to the fluid receiver 30. Specifically, in this example, the first fluid source 10 is a drug source 12 in the form of a drug vial and the fluid receiver 30 is a medication bag 32. Operation of the medical fluid transfer system 300 thus can include transferring a subset of the contents of the drug source 12 into the medication bag 32, which in this example may be pre-filled with a volume of diluent ingredients. It is to be understood that other examples of the first fluid source 10 (e.g., of the drug source 12) and/or the fluid receiver 30 can be used in the example of FIG. 3.

The first channel pump 320 can operate to convey the first fluid 11 from the first fluid source 10 to the fluid receiver 30 in a manner at least substantially similar to that described above with reference to the first channel pump 120 of FIG. 1 and/or the first channel pump 220 of FIGS. 2A-2B.

As shown in FIG. 3, the medical fluid transfer system 300 additionally may include an optional second channel pump 340, which may represent an example of the second channel pump 140 of FIG. 1 and/or the second channel pump 240 of FIG. 2A. The second channel pump 340 may optionally be used to transfer a second fluid from a second fluid source to the fluid receiver 30 in the manner described above with reference to FIG. 1. Additionally, or alternatively, the second channel pump 340 optionally may be used to transfer a second fluid from a second fluid source to a second fluid receiver, thereby mirroring the operation of the first channel pump 320 to perform two independent fluid transfer operations. As another example, the first channel pump 320 and the second channel pump 340 can operate cooperatively to transfer the first fluid 11 to the fluid receiver 30 from a common first fluid source 10 and/or from two separate fluid sources each containing the same first fluid 11, such as in the manner described above with reference to FIG. 2A.

In this manner, an example of the medical fluid transfer system 300 including each of the first channel pump 320 and the second channel pump 340 may be at least substantially similar and/or identical in structure, form, and/or function to the medical fluid transfer system 100 and/or the medical fluid transfer system 200. Stated differently, FIG. 3 may be described as depicting an optional mode of operation of a medical fluid transfer system that includes two channels (e.g., the medical fluid transfer system 100 and/or the medical fluid transfer system 200) in which only one of the channels is used in a fluid transfer procedure.

Specifically, FIG. 3 depicts an example in which only the first channel pump 320 is used to transfer the first fluid 11 from the first fluid source 10 to the fluid receiver 30. Accordingly, in this example, the first channel pump 320 also may be referred to as an active channel pump 320. Similarly, the inlet port 322 of the first channel pump 320 also may be referred to as an active inlet port 322 and/or the outlet port 370 of the first channel pump 320 also may be referred to as an active outlet port 324. In such an example, the second channel pump 340 also may be referred to as an idle channel pump 340 with an idle pump inlet and and idle pump outlet.

While FIG. 3 depicts an example in which the first channel pump 320 is used and the second channel pump 340 is unused (e.g., idle), it also is within the scope of the present disclosure that the second channel pump 340 alone may be used (e.g., with the first channel pump 320 idle) in a fluid transfer procedure in a similar manner.

As shown in FIG. 3, the first fluid source 10 is fluidly connected to the inlet port 322 of the first channel pump 320, and the outlet port 324 of the first channel pump 320 is connected to fluid receiver 30 via a connection port 370 (e.g., without a corresponding first fluid conduit. The outlet port 324 and/or the connection port 370 can be connected directly to the fluid receiver 30, such as via an ISO standard Luer based connection.

In an example of using the medical fluid transfer system 300 of FIG. 3, a user (e.g., a clinician) can enter a desired amount of the first fluid 11 into a user interface of the medical fluid transfer system 300 (e.g., the user interface 182 of FIG. 1), optionally following configuration and priming of the medical fluid transfer system 200. In some examples, the user can decline a prompt presented by the user interface to enter a volume of a second fluid to be transferred to the fluid receiver 30. A controller of the medical fluid transfer system 300 (e.g., the controller 180 of FIG. 1) can then operate to actuate the pump driver 332 of the first channel pump 320 to draw the first fluid 11 into the pump reservoir 330 via an intake stroke. Through a series of valves and/or stopcocks (e.g., the valve 326), the direction of the first fluid 11 can be passively and/or actively controlled to route the first fluid 11 into the fluid receiver 30, such as during a subsequent expulsion stroke of the first channel pump 320.

In this manner, the medical fluid transfer system 300 can be used to transfer a single ingredient from the first fluid source 10 to the fluid receiver 30. As discussed above, FIG. 3 thus may be described as representing optional modes of operation of the medical fluid transfer system 100 of FIG. 1 and/or of the medical fluid transfer system 200 of FIG. 2A in which a user can select whether to transfer a single fluid to the fluid receiver 30 with the first channel pump 320 or to transfer two different fluids (e.g., different ingredients) into the fluid receiver 30 via parallel operation of the first channel pump 320 and the second channel pump 340.

Upon completion of such an operation, the fluid receiver 30 will contain an accurate volume of the first fluid 11, thus achieving accuracy for the drug volume and/or total volume in the fluid receiver 30 without relying on pre-filled IV fluid containers.

FIGS. 4A-4B represent examples of a medical fluid transfer system 400 in which the first channel pump 420 operates to convey the first fluid 11 from the first fluid source 10 to the fluid receiver 30 and in which the second channel pump 440 operates to convey the second fluid 21 from the second fluid source 20 to the fluid receiver 30. Specifically, in this example, the first fluid source 10 is a drug source 12 in the form of a drug vial, the second fluid source 20 is a bulk fluid source 14 in the form of an IV bag, and the fluid receiver 30 is a medication bag 32. Operation of the medical fluid transfer system 400 thus can include transferring a subset of the contents of the drug source 12 and a subset of the contents of the bulk fluid source 14 into the medication bag 32. It is to be understood that other examples of the first fluid source 10 (e.g., of the drug source 12), of the second fluid source (e.g., of the bulk fluid source 14), and/or the fluid receiver 30 can be used in the example of FIG. 4.

The medical fluid transfer system 400 of FIGS. 4A-4B can share any suitable structures, features, and/or functionalities with the medical fluid transfer system 100 of FIG. 1, the medical fluid transfer system 200 of FIG. 2A, and/or the medical fluid transfer system 300 of FIG. 3. Additionally, or alternatively, FIGS. 4A-4B may be described as depicting an optional mode of operation of the medical fluid transfer system 100 of FIG. 1, the medical fluid transfer system 200 of FIG. 2A, and/or the medical fluid transfer system 300 of FIG. 3. Similarly, FIG. 2A and/or FIG. 3 additionally or alternatively may be described as depicting an optional mode of operation of the medical fluid transfer system 400 of FIGS. 4A-4B.

The first channel pump 420 can operate to convey the first fluid 11 from the first fluid source 10 to the fluid receiver 30 in a manner at least substantially similar to that described above with reference to the first channel pump 120 of FIG. 1, the first channel pump 220 of FIGS. 2A-2B, and/or the first channel pump 320 of FIG. 3. Similarly, the second channel pump 440 can operate to convey the second fluid 21 from the second fluid source 20 to the fluid receiver 30 in a manner at least substantially similar to that described above with reference to the second channel pump 140 of FIG. 1, the second channel pump 240 of FIG. 2A, and/or the second channel pump 340 of FIG. 3. In particular, the medical fluid transfer system 400 of FIG. 4 may be described as representing an optional mode of operation of the medical fluid transfer system 300 of FIG. 3 in which the medical fluid transfer system 300 includes the second channel pump 340.

As shown in FIG. 4A, the first fluid source 10 is fluidly connected to the inlet port 422 of the first channel pump 420 and the second fluid source 20 is fluidly connected to the inlet port 442 of the second channel pump 440. Similar to the medical fluid transfer system 200 of FIG. 2A, the operation of the first channel pump 420 and the second channel pump 440 can be staggered such that the first channel pump 420 draws the first fluid 11 from the first fluid source 10 through the inlet port 422 while the second channel pump 440 expels the first fluid 11 out of the outlet port 444 and into the fluid receiver 30. Stated differently, the first channel pump 420 and the second channel pump 440 can be operated such that the first channel pump 420 performs the intake stroke while the second channel pump 440 performs the expulsion stroke, and vice versa. As a result, the fluid receiver 30 may be continuously (or nearly continuously) filled with the first fluid 11 and/or the second fluid 21 during a fluid transfer procedure.

In the example of FIG. 4A, the outlet port 424 of the first channel pump 420 is connected to the conduit junction 464 via a corresponding connection port 470 (e.g., without a corresponding first fluid conduit), while the outlet port 444 of the second channel pump 440 is connected to the conduit junction 464 via a corresponding connection port 470 and the second fluid conduit 462. The conduit junction 464 can be connected directly to the fluid receiver 30, such as via an ISO standard Luer based connection.

The example of FIG. 4B is substantially similar to the example of FIG. 4A with the exception that the example of FIG. 4B lacks the conduit junction 464 of FIG. 4A. Instead, in the example of FIG. 4B, the outlet port 424 of the first channel pump 420 is connected to the fluid receiver 30 via a corresponding connection port 470 and the first fluid conduit 460, while the outlet port 444 of the second channel pump 440 is connected to a separate port of the fluid receiver 30 via a corresponding connection port 470 and the second fluid conduit 462. Operation of the medical fluid transfer systems 400 of FIGS. 4A-4B is otherwise identical.

In an example of using the medical fluid transfer system 400 of FIG. 4A or FIG. 4B, a user (e.g., a clinician) can enter a desired amount of the first fluid 11 and a desired amount of the second fluid 21 into a user interface of the medical fluid transfer system 400 (e.g., the user interface 182 of FIG. 1), optionally following configuration and priming of the medical fluid transfer system 400. A controller of the medical fluid transfer system 400 (e.g., the controller 180 of FIG. 1) can then operate to actuate the pump driver 432 of the first channel pump 420 to draw the first fluid 11 into the pump reservoir 230 via an intake stroke and to subsequently convey the first fluid 11 to the fluid receiver 30 via an expulsion stroke. Similarly, the controller can operate to actuate the pump driver 452 of the second channel pump 440 to draw the second fluid 21 into the pump reservoir 450 via an intake stroke and to subsequently convey the second fluid 21 to the fluid receiver 30 via an expulsion stroke. Through a series of valves and/or stopcocks (e.g., the valves 426, 446), the direction of the first fluid 11 and the second fluid 21 can be passively and/or actively controlled to route the first fluid 11 and the second fluid 21 into the fluid receiver 30.

While the first channel pump 420 operates to transfer the first fluid 11 into the fluid receiver 30 (e.g., with an expulsion stroke), the second channel pump 440 can begin to withdraw the second fluid 21 into the pump reservoir 450 from the second fluid source 20 (e.g., with an intake stroke). In other words, while the first channel pump 420 is transferring the first fluid 11 to the fluid receiver 30, the second channel pump 440 will simultaneously withdraw the second fluid 21 from the second fluid source 20.

Similarly, while the second channel pump 440 subsequently operates to transfer the second fluid 21 into the fluid receiver 30 (e.g., with an expulsion stroke), the first channel pump 420 can begin to withdraw the first fluid 11 into the pump reservoir 430 from the first fluid source 10 (e.g., with an intake stroke). In other words, while the second channel pump 440 is transferring the second fluid 21 to the fluid receiver 30, the first channel pump 420 will simultaneously withdraw the first fluid 11 from the first fluid source 10.

In some examples, the first channel pump 420 transfers the first fluid 11 to the fluid receiver 30 at the same rate, or at a similar rate, as the second channel pump 440 transfers the second fluid 21 to the fluid receiver 30. Rates may be described as being similar to one another when the rates are within +/−10% of each other. In other examples, the rate at which the first channel pump 420 transfers the first fluid 11 to the fluid receiver 30 may be different from the rate at which the second channel pump 440 transfers the second fluid 21 to the fluid receiver 30.

Upon completion of such an operation, the fluid receiver 30 will contain an accurate volume of each of the first fluid 11 and the second fluid 21, thus achieving accuracy for the total volume of the resulting fluid formulation in the fluid receiver 30 without relying on pre-filled IV fluid containers.

As discussed above, the medical fluid transfer system 400 can include one or more (e.g., a network of) valves and/or stopcocks, such as the valve 426 and the valve 446, to ensure that the first channel pump 420 can transfer the first fluid 11 and the second channel pump 440 can transfer the second fluid 21 as described herein. For example, and as discussed above with reference to FIG. 1, the medical fluid transfer system 400 can include one or more check valves positioned and/or oriented to restrict and/or prevent a fluid flow in a direction other than those described herein.

### ADDITIONAL EXAMPLES OF THE DISCLOSED TECHNOLOGY

Having described and illustrated the principles of the disclosed technology with reference to the illustrated examples, it will be recognized that the illustrated examples can be modified in arrangement and detail without departing from such principles. For instance, elements of examples performed in software may be implemented in hardware and vice-versa. Also, the technologies from any example can be combined with the technologies described in any one or more of the other examples. It will be appreciated that procedures and functions such as those described with reference to the illustrated examples can be implemented in a single hardware or software module, or separate modules can be provided. The particular arrangements above are provided for convenient illustration, and other arrangements can be used.

Example 1. A medical fluid transfer system, comprising: a base; a first channel pump supported by the base and comprising a first inlet port and a first outlet port; a second channel pump supported by the base and comprising a second inlet port and a second outlet port; and a controller programmed to at least partially control operation of the medical fluid transfer system, wherein each of the first inlet port and the second inlet port are configured to be fluidly connected to a fluid source containing a medical fluid, wherein each of the first channel pump and the second channel pump are configured to convey a subset of the medical fluid from the fluid source to a fluid receiver that is fluidly coupled to each of the first outlet port and the second outlet port, and wherein the controller is programmed to control operation of each of the first channel pump and the second channel pump such that: (i) the first channel pump withdraws the medical fluid from the fluid source while the second channel pump transfers the medical fluid into the fluid receiver; and (ii) the second channel pump withdraws the medical fluid from the fluid source while the first channel pump transfers the medical fluid into the fluid receiver.

Example 2. The medical fluid transfer system of any example herein, particularly example 1, wherein the first channel pump comprises: a first cassette comprising a first pump reservoir; a first plunger body; and a first pump driver configured to drive the first pump driver within the first pump reservoir to vary a volume of the first pump reservoir, wherein the second channel pump comprises: a second cassette comprising a second pump reservoir; a second plunger body; and a second pump driver configured to drive the second pump driver within the second pump reservoir to vary a volume of the second pump reservoir, and wherein the controller is programmed to control each of the first pump driver and the second pump driver such that: (i) the first pump driver operates to increase the volume of the first pump reservoir while the second pump driver operates to decrease the volume of the second pump reservoir; and (ii) the first pump driver operates to decrease the volume of the first pump reservoir while the second pump driver operates to increase the volume of the second pump reservoir.

Example 3. The medical fluid transfer system of any example herein, particularly any one of examples 1-2, wherein the first channel pump comprises a first valve that operates to restrict a flow of the medical fluid through the first inlet port toward the fluid source and to restrict a flow of the medical fluid through the first outlet port away from the fluid receiver, and wherein the second channel pump comprises a second valve that operates to restrict a flow of the medical fluid through the second inlet port toward the fluid source and to restrict a flow of the medical fluid through the second outlet port away from the fluid receiver.

Example 4. The medical fluid transfer system of any example herein, particularly example 3, wherein one or both of the first valve and the second valve operates to passively direct a flow of the medical fluid.

Example 5. The medical fluid transfer system of any example herein, particularly any one of examples 3-4, wherein the controller is programmed to control operation of one or both of the first valve and the second valve to direct a flow of the medical fluid.

Example 6. The medical fluid transfer system of any example herein, particularly any one of examples 1-5, further comprising a conduit junction configured to merge a flow of the medical fluid exiting the first outlet port and a flow of the medical fluid exiting the second outlet port, wherein the conduit junction is positioned upstream of the fluid receiver.

Example 7. The medical fluid transfer system of any example herein, particularly any one of examples 1-6, wherein the second outlet port is fluidly connected to the fluid receiver via a second fluid conduit extending at least partially exterior of the base.

Example 8. The medical fluid transfer system of any example herein, particularly any one of examples 1-7, wherein one or both of the first channel pump and the second channel pump are at least partially received within the base.

Example 9. The medical fluid transfer system of any example herein, particularly any one of examples 1-8, wherein one or both of the first channel pump and the second channel pump are configured to be selectively and repeatedly removed from and coupled to the base without damage to the medical fluid transfer system.

Example 10. The medical fluid transfer system of any example herein, particularly any one of examples 1-9, wherein the controller comprises a user interface configured to receive instructions from a user and to provide instructions to the user, wherein the user interface is at least partially supported by the base.

Example 11. A method of operating a medical fluid transfer system to transfer a medical fluid from a fluid source to a fluid receiver, the method comprising: withdrawing the medical fluid from the fluid source into a first channel pump of the medical fluid transfer system; transferring the medical fluid from the first channel pump to the fluid receiver; withdrawing the medical fluid from the fluid source into a second channel pump of the medical fluid transfer system; and transferring the medical fluid from the second channel pump to the fluid receiver, wherein the withdrawing the medical fluid from the fluid source into the first channel pump and the transferring the medical fluid from the second channel pump to the fluid receiver are performed at least partially concurrently, wherein the withdrawing the medical fluid from the fluid source into the second channel pump and the transferring the medical fluid from the first channel pump to the fluid receiver are performed at least partially concurrently, and wherein the method comprises transferring a predetermined volume of the medical fluid contained within the fluid source to the fluid receiver.

Example 12. The method of any example herein, particularly example 11, wherein the withdrawing the medical fluid from the fluid source into the first channel pump comprises operating a first pump driver of the first channel pump to perform a first intake stroke, wherein the transferring the medical fluid from the first channel pump to the fluid receiver comprises operating the first pump driver to perform a first expulsion stroke, wherein the withdrawing the medical fluid from the fluid source into the second channel pump comprises operating a second pump driver of the second channel pump to perform a second intake stroke, and wherein the transferring the medical fluid from the second channel pump to the fluid receiver comprises operating the second pump driver to perform a second expulsion stroke.

Example 13. The method of any example herein, particularly example 12, wherein the operating the first pump driver to perform the first intake stroke and the first expulsion stroke comprises transmitting, with a controller of the medical fluid transfer system, a first pump control signal to the first pump driver, wherein the operating the second pump driver to perform the second intake stroke and the second expulsion stroke comprises transmitting, with the controller, a second pump control signal to the second pump driver, and wherein the method comprises generating, with the controller, each of the first pump control signal and the second pump control signal such that the first pump driver performs the first intake stroke while the second pump driver performs the second expulsion stroke and such that the first pump driver performs the first expulsion stroke while the second pump driver performs the second intake stroke.

Example 14. The method of any example herein, particularly any one of examples 11-13, further comprising: receiving, via a user interface of the medical fluid transfer system, a user input indicating a predetermined total volume of the medical fluid to be transferred from the fluid source to the fluid receiver; and controlling, with a controller of the medical fluid transfer system, operation of the first channel pump and the second channel pump to transfer the predetermined total volume of the medical fluid from the fluid source to the fluid receiver.

Example 15. The method of any example herein, particularly any one of examples 11-14, further comprising, prior to the withdrawing the medical fluid from the fluid source: fluidly coupling the fluid source to an inlet port of the medical fluid transfer system; and fluidly coupling the fluid receiver to an outlet port of the medical fluid transfer system, and wherein the method further comprises, subsequent to the transferring the predetermined volume of the medical fluid to the fluid receiver: removing the fluid source from the inlet port; and removing the fluid receiver from the outlet port such that the medical fluid may be administered to a patient from the fluid receiver.

Example 16. The method of any example herein, particularly example 15, further comprising administering the medical fluid to the patient intravenously.

Example 17. The method of any example herein, particularly any one of examples 11-16, wherein the transferring the medical fluid from the first channel pump to the fluid receiver comprises transferring the medical fluid at a first flow rate, and wherein the transferring the medical fluid from the second channel pump to the fluid receiver comprises transferring the medical fluid at a second flow rate that is similar to the first flow rate.

Example 18. The method of any example herein, particularly any one of examples 11-17, wherein the transferring the medical fluid from the first channel pump to the fluid receiver comprises transferring the medical fluid at a first flow rate, wherein the transferring the medical fluid from the second channel pump to the fluid receiver comprises transferring the medical fluid at a second flow rate, and wherein the method further comprises receiving, with a user interface of the medical fluid transfer system, a user input specifying one or both of the first flow rate and the second flow rate.

Example 19. The method of any example herein, particularly any one of examples 11-18, further comprising: receiving, with a controller of the medical fluid transfer system, an input fluid signal including information regarding one or more of an identification of the medical fluid, a lot number corresponding to the medical fluid, or an expiration date of the medical fluid; and recording, with the controller, fluid transfer information that represents one or both of the identification of the medical fluid transferred to the fluid receiver and an amount of medical fluid transferred to the fluid receiver, wherein the fluid transfer information is based, at least in part, on the input fluid signal.

Example 20. The method of any example herein, particularly example 19, further comprising receiving, with the controller, an output fluid signal including information regarding the amount of medical fluid transferred to the fluid receiver, and wherein the fluid transfer information is based, at least in part, on the output fluid signal.

Example 21. The method of any example herein, particularly any one of examples 11-20, wherein the medical fluid transfer system is the medical fluid transfer system of any example herein, particularly any one of examples 1-10.

Example 22. A medical fluid transfer system, comprising: a base; a pump supported by the base and comprising an inlet port and an outlet port; and a controller programmed to at least partially control operation of the medical fluid transfer system, wherein the inlet port is configured to be fluidly connected to a fluid source containing a medical fluid, wherein the pump is configured to convey a subset of the medical fluid from the fluid source to a fluid receiver that is fluidly coupled to the outlet port, wherein the pump comprises a cassette comprising a pump reservoir, a plunger body, and a pump driver configured to drive the pump driver within the pump reservoir to vary a volume of the pump reservoir, wherein the base comprises a pump receiver that receives at least a portion of the pump within the base, wherein the medical fluid transfer system is configured such that the cassette may be selectively and repeatedly removed from and received within the pump receiver without damage to the medical fluid transfer system, and wherein the controller is configured to transmit a pump control signal to the pump driver to control operation of the pump based, at least in part, on a user input specifying a volume of the medical fluid to be transferred to the fluid receiver.

Example 23. The medical fluid transfer system of any example herein, particularly example 22, wherein the fluid source is a first fluid source, wherein the medical fluid is a first medical fluid, wherein the pump is a first channel pump, wherein the inlet port is a first inlet port, wherein the outlet port is a first outlet port, wherein the cassette is a first cassette, wherein the pump reservoir is a first pump reservoir, wherein the plunger body is a first plunger body, wherein the pump driver is a first pump driver, wherein the pump control signal is a first pump control signal, and wherein the medical fluid transfer system further comprises a second channel pump supported by the base, wherein the second channel pump comprises: a second inlet port; a second outlet port; and a second cassette with a second pump reservoir, a second plunger body, and a second pump driver configured to drive the second pump driver within the second pump reservoir to vary a volume of the second pump reservoir, wherein the second inlet port is configured to be fluidly connected to a second fluid source containing a second medical fluid, wherein the second channel pump is configured to convey a subset of the second medical fluid from the second fluid source to a second fluid receiver that is fluidly coupled to the second outlet port, and wherein the controller is configured to transmit a second pump control signal to the second pump driver to control operation of the first channel pump based, at least in part, on a user input specifying a volume of the second medical fluid to be transferred to the second fluid receiver.

Example 24. The medical fluid transfer system of any example herein, particularly any one of examples 22-23, wherein the pump comprises a valve that operates to restrict a flow of the medical fluid through the inlet port toward the fluid source and to restrict a flow of the medical fluid through the outlet port away from the fluid receiver.

Example 25. The medical fluid transfer system of any example herein, particularly example 24, wherein the valve operates to passively direct a flow of the medical fluid.

Example 26. The medical fluid transfer system of any example herein, particularly any one of examples 24-25, wherein the controller is programmed to control operation of the valve to direct a flow of the medical fluid.

Example 27. The medical fluid transfer system of any example herein, particularly any one of examples 22-26, further comprising a fluid conduit extending between the outlet port and the fluid receiver to convey the medical fluid from the outlet port to the fluid receiver, and wherein the fluid conduit extends at least partially exterior of the base.

Example 28. The medical fluid transfer system of any example herein, particularly any one of examples 22-27, further comprising a connection port attached to the base, wherein the connection port is configured to be directly coupled to the fluid receiver to convey the medical fluid to the fluid receiver via the connection port.

Example 29. The medical fluid transfer system of any example herein, particularly any one of examples 22-28, wherein the pump is fully enclosed within the base during operative use of the medical fluid transfer system.

Example 30. The medical fluid transfer system of any example herein, particularly any one of examples 22-29, wherein the controller comprises a user interface configured to receive the user input from a user and to provide instructions to the user, wherein the user interface is at least partially supported by the base.

Example 31. The medical fluid transfer system of any example herein, particularly example 30, wherein the user interface comprises one or both of a display device and a user input device.

Example 32. A method of operating a medical fluid transfer system to transfer a medical fluid from a fluid source to a fluid receiver, the method comprising: receiving, with a controller of the medical fluid transfer system, a user input representing a predetermined volume of the medical fluid to be transferred from the fluid source to the fluid receiver; and transferring the predetermined volume of the medical fluid from the fluid source to the fluid receiver, wherein the transferring the predetermined volume comprises repeating the steps of: withdrawing the medical fluid from the fluid source into a pump reservoir of a pump of the medical fluid transfer system; and expelling the medical fluid from the pump reservoir to transfer the medical fluid to the fluid receiver, wherein each of the withdrawing the medical fluid from the fluid source into the pump reservoir and the expelling the medical fluid from pump reservoir is performed responsive to a pump control signal transmitted from the controller to the pump, and wherein the method comprises generating, with the controller, the pump control signal based, at least in part, on the user input.

Example 33. The method of any example herein, particularly example 32, wherein the withdrawing the medical fluid from the fluid source into the pump reservoir comprises operating a pump driver of the pump to move a plunger body of the pump within the pump reservoir to perform an intake stroke, and wherein the expelling the medical fluid from the pump reservoir comprises operating the pump driver to move the plunger body within the pump reservoir to perform an expulsion stroke.

Example 34. The method of any example herein, particularly any one of examples 32-33, wherein the transferring the predetermined volume of the medical fluid comprises repeating the steps of withdrawing the medical fluid from the fluid source and expelling the medical fluid form the pump reservoir in a predetermined number of iterations, and wherein the method comprises, prior to the transferring the predetermined volume of the medical fluid, determining the predetermined number of iterations based, at least in part, on the predetermined volume.

Example 35. The method of any example herein, particularly any one of examples 32-34, wherein the receiving the user input comprises receiving via a user interface of the medical fluid transfer system that is at least partially supported by a base of the medical fluid transfer system.

Example 36. The method of any example herein, particularly any one of examples 32, further comprising, prior to the withdrawing the medical fluid from the fluid source: fluidly coupling the fluid source to an inlet port of the medical fluid transfer system; and fluidly coupling the fluid receiver to an outlet port of the medical fluid transfer system, and wherein the method further comprises, subsequent to the transferring the predetermined volume of the medical fluid to the fluid receiver: removing the fluid source from the inlet port; and removing the fluid receiver from the outlet port such that the medical fluid may be administered to a patient from the fluid receiver.

Example 37. The method of any example herein, particularly example 36, wherein one or both of the fluidly coupling the fluid source to the inlet port and the fluidly coupling the fluid receiver to the outlet port comprises coupling via an ISO standard Luer based coupling.

Example 38. The method of any example herein, particularly any one of examples 36-37, further comprising administering the medical fluid to the patient intravenously.

Example 39. The method of any example herein, particularly any one of examples 32-38, wherein the user input further includes information specifying a predetermined flow rate, and wherein the transferring the predetermined volume comprises transferring the medical fluid into the fluid receiver at the predetermined flow rate.

Example 40. The method of any example herein, particularly any one of examples 32-39, further comprising: receiving, with the controller, an input fluid signal including information regarding one or more of an identification of the medical fluid, a lot number corresponding to the medical fluid, or an expiration date of the medical fluid; and recording, with the controller, fluid transfer information that represents one or both of the identification of the medical fluid transferred to the fluid receiver and an amount of medical fluid transferred to the fluid receiver, wherein the fluid transfer information is based, at least in part, on the input fluid signal.

Example 41. The method of any example herein, particularly example 40, further comprising receiving, with the controller, an output fluid signal including information regarding the amount of medical fluid transferred to the fluid receiver, and wherein the fluid transfer information is based, at least in part, on the output fluid signal.

Example 42. The method of any example herein, particularly any one of examples 32-41, wherein the medical fluid transfer system is the medical fluid transfer system of any example herein, particularly any one of examples 22-31.

Example 43. A medical fluid transfer system, comprising: a base; a first channel pump supported by the base and comprising a first inlet port and a first outlet port; a second channel pump supported by the base and comprising a second inlet port and a second outlet port; and a controller programmed to at least partially control operation of the medical fluid transfer system, wherein the first inlet port is configured to be fluidly coupled to a first fluid source containing a first medical fluid, wherein the second inlet port is configured to be fluidly coupled to a second fluid source containing a second medical fluid, wherein the first channel pump is configured to convey a subset of the first medical fluid from the first fluid source to a fluid receiver that is fluidly coupled to each of the first outlet port and the second outlet port, wherein the second channel pump is configured to convey a subset of the second medical fluid from the second fluid source to the fluid receiver, and wherein the controller is programmed to control operation of the medical fluid transfer system such that, in a first mode of operation, the controller at least partially controls operation of each of the first channel pump and the second channel pump such that the first channel pump conveys a first predetermined volume of the first medical fluid from the first fluid source to the fluid receiver and such that the second channel pump conveys a second predetermined volume of the second medical fluid from the second fluid source to the fluid receiver; wherein the first channel pump comprises: a first cassette comprising a first pump reservoir; a first plunger body; and a first pump driver configured to drive the first pump driver within the first pump reservoir to vary a volume of the first pump reservoir, wherein the second channel pump comprises: a second cassette comprising a second pump reservoir; a second plunger body; and a second pump driver configured to drive the second pump driver within the second pump reservoir to vary a volume of the second pump reservoir.

Example 44. The medical fluid transfer system of any example herein, particularly example 43, wherein the controller further is programmed to control operation of the medical fluid transfer system such that, in a second mode of operation, the controller at least partially controls operation of only one of the first channel pump or the second channel pump such that only one of: (i) the first channel pump conveys a first predetermined volume of the first medical fluid from the first fluid source to the fluid receiver; or (ii) that the second channel pump conveys a second predetermined volume of the second medical fluid from the second fluid source to the fluid receiver.

Example 45. The medical fluid transfer system of any example herein, particularly any one of examples 43-44, wherein the controller is programmed to control each of the first pump driver and the second pump driver such that, in the first mode of operation: (i) the first pump driver operates to increase the volume of the first pump reservoir while the second pump driver operates to decrease the volume of the second pump reservoir; and (ii) the first pump driver operates to decrease the volume of the first pump reservoir while the second pump driver operates to increase the volume of the second pump reservoir.

Example 46. The medical fluid transfer system of any example herein, particularly any one of examples 43-45, wherein the first channel pump comprises a first valve that operates to restrict a flow of the first medical fluid through the first inlet port toward the first fluid source and to restrict a flow of the first medical fluid through the first outlet port away from the fluid receiver, and wherein the second channel pump comprises a second valve that operates to restrict a flow of the second medical fluid through the second inlet port toward the second fluid source and to restrict a flow of the second medical fluid through the second outlet port away from the fluid receiver.

Example 47. The medical fluid transfer system of any example herein, particularly example 46, wherein one or both of the first valve and the second valve operates to passively direct a flow of the first medical fluid or the second medical fluid.

Example 48. The medical fluid transfer system of any example herein, particularly any one of examples 46-47, wherein the controller is programmed to control operation of one or both of the first valve and the second valve to direct a flow of the first medical fluid or the second medical fluid.

Example 49. The medical fluid transfer system of any example herein, particularly any one of examples 43-48, further comprising a conduit junction configured to merge a flow of the first medical fluid exiting the first outlet port and a flow of the second medical fluid exiting the second outlet port, wherein the conduit junction is positioned upstream of the fluid receiver.

Example 50. The medical fluid transfer system of any example herein, particularly any one of examples 43-49, wherein the second outlet port is fluidly connected to the fluid receiver via a second fluid conduit extending at least partially exterior of the base.

Example 51. The medical fluid transfer system of any example herein, particularly any one of examples 43-50, wherein one or both of the first channel pump and the second channel pump are at least partially received within the base.

Example 52. The medical fluid transfer system of any example herein, particularly any one of examples 43-51, wherein one or both of the first channel pump and the second channel pump are configured to be selectively and repeatedly removed from and coupled to the base without damage to the medical fluid transfer system.

Example 53. The medical fluid transfer system of any example herein, particularly any one of examples 43-52, wherein the controller comprises a user interface configured to receive instructions from a user and to provide instructions to the user, wherein the user interface is at least partially supported by the base.

Example 54. A method of operating a medical fluid transfer system to transfer a first medical fluid from a first fluid source to a fluid receiver and to transfer a second medical fluid from a second fluid source to the fluid receiver, the method comprising: receiving, with a controller of the medical fluid transfer system, a user input representing each of a first predetermined volume of the first medical fluid to be transferred from the first fluid source to the fluid receiver and a second predetermined volume of the second medical fluid to be transferred from the second fluid source to the fluid receiver; withdrawing the first medical fluid from the first fluid source into a first channel pump of the medical fluid transfer system; transferring the first medical fluid from the first channel pump to the fluid receiver; withdrawing the second medical fluid from the second fluid source into a second channel pump of the medical fluid transfer system; and transferring the second medical fluid from the second channel pump to the fluid receiver, wherein the withdrawing the first medical fluid from the first fluid source into the first channel pump comprises operating a first pump driver of the first channel pump to perform a first intake stroke to draw the first medical fluid into a first pump reservoir of the first channel pump, wherein the transferring the first medical fluid from the first channel pump to the fluid receiver comprises operating the first pump driver to perform a first expulsion stroke to transfer the first medical fluid from the first pump reservoir to the fluid receiver, wherein the withdrawing the second medical fluid from the second fluid source into the second channel pump comprises operating a second pump driver of the second channel pump to perform a second intake stroke to draw the second medical fluid into a second pump reservoir of the second channel pump, and wherein the transferring the second medical fluid from the second channel pump to the fluid receiver comprises operating the second pump driver to perform a second expulsion stroke to transfer the second medical fluid from the second pump reservoir to the fluid receiver.

Example 55. The method of any example herein, particularly example 54, wherein each of the withdrawing the first medical fluid from the first fluid source into the first channel pump and the transferring the first medical fluid from the first channel pump to the fluid receiver is performed responsive to a first pump control signal transmitted from the controller to the first channel pump, wherein each of the withdrawing the second medical fluid from the second fluid source into the second channel pump and the transferring the second medical fluid from the second channel pump to the fluid receiver is performed responsive to a second pump control signal transmitted from the controller to the second channel pump, and wherein the method comprises generating, with the controller, each of the first pump control signal and the second pump control signal based, at least in part, on the user input.

Example 56. The method of any example herein, particularly example 55, wherein the operating the first pump driver to perform the first intake stroke and the first expulsion stroke is performed responsive to the first pump driver receiving the first pump control signal, wherein the operating the second pump driver to perform the second intake stroke and the second expulsion stroke is performed responsive to the second pump driver receiving the second pump control signal, and wherein the method comprises transmitting, with the controller, each of the first pump control signal and the second pump control signal such that the first pump driver performs the first intake stroke while the second pump driver performs the second expulsion stroke and such that the first pump driver performs the first expulsion stroke while the second pump driver performs the second intake stroke.

Example 57. The method of any example herein, particularly any one of examples 54-56, further comprising, prior to the withdrawing the first medical fluid from the first fluid source: fluidly coupling the first fluid source to a first inlet port of the medical fluid transfer system; fluidly coupling the second fluid source to a second inlet port of the medical fluid transfer system; and fluidly coupling the fluid receiver to each of a first outlet port and a second outlet port of the medical fluid transfer system, and wherein the method further comprises, subsequent to the transferring the first predetermined volume of the first medical fluid and the second predetermined volume of the second medical fluid to the fluid receiver: removing the first fluid source from the first inlet port; removing the second fluid source from the second inlet port; and removing the fluid receiver from the medical fluid transfer system such that a fluid formulation comprising the first medical fluid and the second medical fluid may be administered to a patient from the fluid receiver.

Example 58. The method of any example herein, particularly example 57, wherein the first medical fluid comprises a drug to be administered intravenously to the patient, wherein the second medical fluid comprises a diluent ingredient, and wherein the method further comprises administering the fluid formulation to the patient intravenously.

Example 59. The method of any example herein, particularly any one of examples 54-58, wherein the transferring the first medical fluid from the first channel pump to the fluid receiver comprises transferring the first medical fluid at a first flow rate, and wherein the transferring the second medical fluid from the second channel pump to the fluid receiver comprises transferring the second medical fluid at a second flow rate that is similar to the first flow rate.

Example 60. The method of any example herein, particularly any one of examples 54-59, wherein the first medical fluid is different than the second medical fluid.

Example 61. The method of any example herein, particularly any one of examples 54-60, further comprising: receiving, with the controller, an input fluid signal including information regarding one or more of an identification of one or both of the first medical fluid and the second medical fluid, a lot number corresponding to one or both of the first medical fluid and the second medical fluid, or an expiration date of one or both of the first medical fluid and the second medical fluid; and recording, with the controller, fluid transfer information that represents one or more of: (i) the identification of the first medical fluid transferred to the fluid receiver; (ii) the identification of the second medical fluid transferred to the fluid receiver; (iii) an amount of the first medical fluid transferred to the fluid receiver; or (iv) an amount of the second medical fluid transferred to the fluid receiver, and wherein the fluid transfer information is based, at least in part, on the input fluid signal.

Example 62. The method of any example herein, particularly example 61, further comprising receiving, with the controller, an output fluid signal including information regarding one or both of the amount of the first medical fluid transferred to the fluid receiver and the amount of the second medical fluid transferred to the fluid receiver, and wherein the fluid transfer information is based, at least in part, on the output fluid signal.

Example 63. The method of any example herein, particularly any one of examples 54-62, wherein the medical fluid transfer system is the medical fluid transfer system of any example herein, particularly any one of examples 43-53.

Example 64. A method of transferring a medical fluid with a medical fluid transfer system that comprises a base, a first channel pump supported by the base, a second channel pump supported by the base, and a controller programmed to at least partially control operation of the medical fluid transfer system, the method comprising: selecting an active channel pump such that the active channel pump is one of the first channel pump or the second channel pump and such that the other of the first channel pump or the second channel pump is an idle channel pump; fluidly coupling a fluid source containing a medical fluid to an active inlet port of the active channel pump; fluidly coupling a fluid receiver to an active outlet port of the active channel pump; and operating, via the controller, the medical fluid transfer system to transfer a predetermined volume of the medical fluid from the fluid source to the fluid receiver with the active channel pump by: drawing the medical fluid into a pump reservoir of the active channel pump via an intake stroke; transferring the medical fluid out of the pump reservoir and into the fluid receiver via an expulsion stroke; and iteratively repeating the intake stroke and the expulsion stroke until the predetermined volume of the medical fluid is transferred to the fluid receiver.

Example 65. The method of any example herein, particularly example 64, wherein the operating the medical fluid transfer system comprises operating such that the idle channel pump is inactive.

Example 66. The method of any example herein, particularly any one of examples 64-65, wherein the medical fluid transfer system is the medical fluid transfer system of any example herein, particularly any one of examples 43-53.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A medical fluid transfer system, comprising:
a base;
a first channel pump supported by the base and comprising a first inlet port and a first outlet port;
a second channel pump supported by the base and comprising a second inlet port and a second outlet port; and
a controller programmed to at least partially control operation of the medical fluid transfer system,
wherein the first inlet port is configured to be fluidly coupled to a first fluid source containing a first medical fluid,
wherein the second inlet port is configured to be fluidly coupled to a second fluid source containing a second medical fluid,
wherein the first channel pump is configured to convey a subset of the first medical fluid from the first fluid source to a fluid receiver that is fluidly coupled to each of the first outlet port and the second outlet port,
wherein the second channel pump is configured to convey a subset of the second medical fluid from the second fluid source to the fluid receiver, and
wherein the controller is programmed to control operation of the medical fluid transfer system such that, in a first mode of operation, the controller at least partially controls operation of each of the first channel pump and the second channel pump such that the first channel pump conveys a first predetermined volume of the first medical fluid from the first fluid source to the fluid receiver and such that the second channel pump conveys a second predetermined volume of the second medical fluid from the second fluid source to the fluid receiver,
wherein the first channel pump comprises:
a first cassette comprising a first pump reservoir;
a first plunger body; and
a first pump driver configured to drive the first pump driver within the first pump reservoir to vary a volume of the first pump reservoir, and
wherein the second channel pump comprises:
a second cassette comprising a second pump reservoir;
a second plunger body; and
a second pump driver configured to drive the second pump driver within the second pump reservoir to vary a volume of the second pump reservoir.

2. The medical fluid transfer system of claim 1, wherein the controller further is programmed to control operation of the medical fluid transfer system such that, in a second mode of operation, the controller at least partially controls operation of only one of the first channel pump or the second channel pump such that only one of:
(i) the first channel pump conveys a first predetermined volume of the first medical fluid from the first fluid source to the fluid receiver; or
(ii) that the second channel pump conveys a second predetermined volume of the second medical fluid from the second fluid source to the fluid receiver.

3. The medical fluid transfer system of claim 1, wherein the controller is programmed to control each of the first pump driver and the second pump driver such that, in the first mode of operation:
(i) the first pump driver operates to increase the volume of the first pump reservoir while the second pump driver operates to decrease the volume of the second pump reservoir; and
(ii) the first pump driver operates to decrease the volume of the first pump reservoir while the second pump driver operates to increase the volume of the second pump reservoir.

4. The medical fluid transfer system of claim 1, wherein the first channel pump comprises a first valve that operates to restrict a flow of the first medical fluid through the first inlet port toward the first fluid source and to restrict a flow of the first medical fluid through the first outlet port away from the fluid receiver, and wherein the second channel pump comprises a second valve that operates to restrict a flow of the second medical fluid through the second inlet port toward the second fluid source and to restrict a flow of the second medical fluid through the second outlet port away from the fluid receiver.

5. The medical fluid transfer system of claim 4, wherein one or both of the first valve and the second valve operates to passively direct a flow of the first medical fluid or the second medical fluid.

6. The medical fluid transfer system of claim 4, wherein the controller is programmed to control operation of one or both of the first valve and the second valve to direct a flow of the first medical fluid or the second medical fluid.

7. The medical fluid transfer system of claim 1, further comprising a conduit junction configured to merge a flow of the first medical fluid exiting the first outlet port and a flow of the second medical fluid exiting the second outlet port, wherein the conduit junction is positioned upstream of the fluid receiver.

8. The medical fluid transfer system of claim 1, wherein the second outlet port is fluidly connected to the fluid receiver via a second fluid conduit extending at least partially exterior of the base.

9. The medical fluid transfer system of claim 1, wherein one or both of the first channel pump and the second channel pump are at least partially received within the base.

10. The medical fluid transfer system of claim 1, wherein one or both of the first channel pump and the second channel pump are configured to be selectively and repeatedly removed from and coupled to the base without damage to the medical fluid transfer system.

11. The medical fluid transfer system of claim 1, wherein the controller comprises a user interface configured to receive instructions from a user and to provide instructions to the user, wherein the user interface is at least partially supported by the base.

12. A method of operating a medical fluid transfer system to transfer a first medical fluid from a first fluid source to a fluid receiver and to transfer a second medical fluid from a second fluid source to the fluid receiver, the method comprising:
receiving, with a controller of the medical fluid transfer system, a user input representing each of a first predetermined volume of the first medical fluid to be transferred from the first fluid source to the fluid receiver and a second predetermined volume of the second medical fluid to be transferred from the second fluid source to the fluid receiver;
withdrawing the first medical fluid from the first fluid source into a first channel pump of the medical fluid transfer system;
transferring the first medical fluid from the first channel pump to the fluid receiver;
withdrawing the second medical fluid from the second fluid source into a second channel pump of the medical fluid transfer system; and
transferring the second medical fluid from the second channel pump to the fluid receiver,
wherein the withdrawing the first medical fluid from the first fluid source into the first channel pump comprises operating a first pump driver of the first channel pump to perform a first intake stroke to draw the first medical fluid into a first pump reservoir of the first channel pump,
wherein the transferring the first medical fluid from the first channel pump to the fluid receiver comprises operating the first pump driver to perform a first expulsion stroke to transfer the first medical fluid from the first pump reservoir to the fluid receiver,
wherein the withdrawing the second medical fluid from the second fluid source into the second channel pump comprises operating a second pump driver of the second channel pump to perform a second intake stroke to draw the second medical fluid into a second pump reservoir of the second channel pump, and
wherein the transferring the second medical fluid from the second channel pump to the fluid receiver comprises operating the second pump driver to perform a second expulsion stroke to transfer the second medical fluid from the second pump reservoir to the fluid receiver.

13. The method of claim 12, wherein each of the withdrawing the first medical fluid from the first fluid source into the first channel pump and the transferring the first medical fluid from the first channel pump to the fluid receiver is performed responsive to a first pump control signal transmitted from the controller to the first channel pump,
wherein each of the withdrawing the second medical fluid from the second fluid source into the second channel pump and the transferring the second medical fluid from the second channel pump to the fluid receiver is performed responsive to a second pump control signal transmitted from the controller to the second channel pump, and
wherein the method comprises generating, with the controller, each of the first pump control signal and the second pump control signal based, at least in part, on the user input.

14. The method of claim 13, wherein the operating the first pump driver to perform the first intake stroke and the first expulsion stroke is performed responsive to the first pump driver receiving the first pump control signal, wherein the operating the second pump driver to perform the second intake stroke and the second expulsion stroke is performed responsive to the second pump driver receiving the second pump control signal, and wherein the method comprises transmitting, with the controller, each of the first pump control signal and the second pump control signal such that the first pump driver performs the first intake stroke while the second pump driver performs the second expulsion stroke and such that the first pump driver performs the first expulsion stroke while the second pump driver performs the second intake stroke.

15. The method of claim 12, further comprising, prior to the withdrawing the first medical fluid from the first fluid source:
fluidly coupling the first fluid source to a first inlet port of the medical fluid transfer system;
fluidly coupling the second fluid source to a second inlet port of the medical fluid transfer system; and
fluidly coupling the fluid receiver to each of a first outlet port and a second outlet port of the medical fluid transfer system, and
wherein the method further comprises, subsequent to the transferring the first predetermined volume of the first medical fluid and the second predetermined volume of the second medical fluid to the fluid receiver:
removing the first fluid source from the first inlet port;
removing the second fluid source from the second inlet port; and
removing the fluid receiver from the medical fluid transfer system such that a fluid formulation comprising the first medical fluid and the second medical fluid may be administered to a patient from the fluid receiver.

16. The method of claim 15, wherein the first medical fluid comprises a drug to be administered intravenously to the patient, wherein the second medical fluid comprises a diluent ingredient, and wherein the method further comprises administering the fluid formulation to the patient intravenously.

17. The method of claim 12, wherein the transferring the first medical fluid from the first channel pump to the fluid receiver comprises transferring the first medical fluid at a first flow rate, and wherein the transferring the second medical fluid from the second channel pump to the fluid receiver comprises transferring the second medical fluid at a second flow rate that is similar to the first flow rate.

18. The method of claim 12, wherein the first medical fluid is different than the second medical fluid.

19. The method of claim 12, further comprising:
receiving, with the controller, an input fluid signal including information regarding one or more of an identification of one or both of the first medical fluid and the second medical fluid, a lot number corresponding to one or both of the first medical fluid and the second medical fluid, or an expiration date of one or both of the first medical fluid and the second medical fluid; and
recording, with the controller, fluid transfer information that represents one or more of:
(i) the identification of the first medical fluid transferred to the fluid receiver;
(ii) the identification of the second medical fluid transferred to the fluid receiver;
(iii) an amount of the first medical fluid transferred to the fluid receiver; or
(iv) an amount of the second medical fluid transferred to the fluid receiver, and wherein the fluid transfer information is based, at least in part, on the input fluid signal.

20. The method of claim 19, further comprising receiving, with the controller, an output fluid signal including information regarding one or both of the amount of the first medical fluid transferred to the fluid receiver and the amount of the second medical fluid transferred to the fluid receiver, and wherein the fluid transfer information is based, at least in part, on the output fluid signal.
